(19) United States
(12) Patent Application Publication
(10) Pub. No.: US 2017/0007725 A1
(43) Pub. Date: Jan. 12, 2017

(54) GOLD NANOSTRUCTURES AND USES THEREOF

(71) Applicant: B. G. Negev Technologies and Applications Ltd., at Ben-Gurion University, Beer Sheva (IL)

(72) Inventors: Luai Khoury, Fassuta (IL); Riki Goldbart, Lehavim (IL); Tamar Traitel, Beer Sheva (IL); Giora Enden, Tel Aviv (IL); Josef Kost, Omer (IL)

(21) Appl. No.: 15/203,897

(22) Filed: Jul. 7, 2016

Related U.S. Application Data
(60) Provisional application No. 62/189,487, filed on Jul. 7, 2015.

(51) Int. Cl. A61K 49/00

(52) U.S. Cl. CPC A61K 49/0093; A61K 49/005; Y10S 977/773; B82Y 15/00

(57) ABSTRACT

Charged nanostructure being comprising gold nanoparticle which may bear on at least portion thereof a positively charged polymer wherein the positively charged polymer may bearon at least portion thereof a negatively charged polymer is disclosed. Uses thereof for diagnosis is also disclosed.

This application claims the benefit of priority from U.S. Provisional Patent Application No. 62/189,487, filed on Jul. 7, 2015. The content of the above document is incorporated by reference in its entirety as if fully set forth herein.

### FIELD OF THE INVENTION

The present invention, in some embodiments thereof, relates to gold nanostructures bearing polymers thereon and to uses thereof and, in harvesting low molecular weight biomarkers.

### BACKGROUND OF THE INVENTION

The efficacy of cancer treatment depends on the pathological state of the disease; the earlier the detection and diagnosis, the more efficient will the treatment be. Several methods are commonly used to detect cancer such as Computed Axial Tomography (CAT) scan, ultrasound and biopsy to mention a few. These methods might lead to false negative diagnosis because of their limited resolution at the early stages of the tumor (CAT scan and ultrasound) or might be risky to the patient because of their invasive nature (biopsy). The lack of early-stage diagnostic tools has stimulated a pursuit for alternative approaches. One of the most promising approaches is based on the fact that tumor cells excrete low molecular weight (LMW) bio-molecules (<10 kDa) to the bloodstream that are specific to the tumor type at a rate proportional to the tumor age/size. Harvesting, concentrating, and analyzing these biomarkers can provide valuable diagnostic information. However, a few drawbacks hinder the immediate and direct utilization of this approach. The major obstacle in selective harvesting of LMW biomarkers from the blood and analyzing them is their low abundance. Proteolysis by blood-plasma proteases further lowers the concentration of these proteins and decreases their detectability. In addition, carrier proteins (e.g., albumin and immunoglobulin (IgG) exist at concentrations billion-fold higher than those of the biomarkers thereby masking the LMW biomarkers and hindering their detection. As a result several sample preparations are needed for mass spectrometry (MS) to filter out the high molecular weight (HMW) proteins. However, recent studies showed that LMW biomarkers are associated non-covalently with HMW protein. Therefore removing HMW proteins from the sample may further dilute the samples from LMW biomarkers and exhibit false negative results (Lowenthal, M. S. et al. Clin. Chem. 2005, 51, 1933).

### SUMMARY OF THE INVENTION

In one embodiment, the present invention provides nanostructure being negatively charged, the nanostructure comprises at least one gold nanoparticle, wherein the at least one gold nanoparticle:

(a) bears on at least portion thereof a positively charged polymer selected from polyethylenamine (PEI), cationic polyallylamine, poly-L-lysine, poly(allylamine hydrochloride), and poly(diallyldimethylammonium chloride), wherein the positively charged polymer bears on at least portion thereof a negatively charged polymer selected from poly(acrylic acid) (PAA), polystyrenesulfonate, polyvinyl sulfate and polyvinylsulfonic acid; and

(b) has an averaged diameter of between 5 and 20 nm,

and wherein the nanostructure has a hydrodynamic diameter of less than 100 nm, or, in some embodiments, less than 25 nm.

In a further embodiment, the positively charged polymer is polyethylenamine (PEI).

In a further embodiment, the negatively charged polymer is poly(acrylic acid) (PAA).

In a further embodiment, the positively charged polymer has an average molecular weight (MW) that ranges from 6 kDa to 15 kDa. In a further embodiment, the negatively charged polymer has an average molecular weight (MW) that ranges from 8 kDa to 15 kDa.

In a further embodiment, the nanostructure is characterized by a zeta-potential of at least |−30| mV in aqueous dispersion. In a further embodiment, the nanostructure is characterized by a high affinity to a positively charged low molecular weight (LMW) molecules.

In a further embodiment, the LMW molecules are selected from peptides, proteins, platelet-derived microparticles, and apoptotic bodies. In a further embodiment, the LMW molecules have a MW value of less than 15 kDa. In a further embodiment, the LMW molecules are selected from Stromal Derived Factor-alpha (SDF-alpha) and Platelet Derived Growth Factor B.

In a further embodiment, the present invention provides composition comprising a plurality of the nanostructures. In a further embodiment, the composition is in form of stable dispersion.

In a further embodiment, at least 90% of the plurality of the nanostructures are characterized by an averaged diameter that varies within ±20%. In a further embodiment, the composition is for use in detection of positively-charged low molecular weight (LMW) molecules.

In a further embodiment, the composition is for use in diagnosis and/or detection of medical disorder or disease, the medical disorder or disease being characterized by occurrence of excessive positively charged low molecular weight (LMW) molecules. In a further embodiment, the composition is for use in diagnosis and/or detection of medical disorder or disease, the medical disorder or disease being characterized by occurrence of excessive negatively charged low molecular weight (LMW) molecules.

In a further embodiment, disease is cancer and/or inflammatory-disease. In a further embodiment, the detection is in a sample of biological cells, in vitro, ex vivo, in vivo, or for clinical imaging. In a further embodiment, the detection is used to characterize the intrinsic apoptotic load within a tumor, the level of aggressiveness of a tumor, or to detect metastases.

In a further embodiment, the composition further comprises a physiologically acceptable carrier. In a further embodiment, the composition further comprises an agent selected from radiolabel, an X-ray contrast agent, a magnetic resonance imaging (MRI) contrast agent, and a fluorescent label. In a further embodiment, the composition is detected by a method selected from fluorescent microscope, a flow-cytometric equipment, MRI contrast, X-ray or computerized tomography (CT) contrast.

In a further embodiment, there is provided method for the detection of positively charged LMW molecules in a sample, the method comprising: contacting the sample with the disclosed composition under conditions enabling binding of the nanostructures to positively-charged LMW molecules; and detecting bound nanostructures to the LMW molecules, thereby indicating the presence of positively-charged LMW molecules. In a further embodiment, the sample is selected from blood serum and blood plasma.

In a further embodiment, the method is performed by a kit. In a further embodiment, the kit comprises a medium having affixed thereto said nanostructure and a detecting assay for detecting LMW molecules.

In a further embodiment, there is provided a method for the detection of a physiological disorder or disease characterized by the presence positively-charged LMW molecules, the method comprising administering the disclosed composition to a patient in need thereof; and imaging the patient using an appropriate imaging technique.

### BRIEF DESCRIPTION OF THE DRAWINGS

Some embodiments of the invention are herein described, by way of example only, with reference to the accompanying drawings. With specific reference now to the drawings in detail, it is stressed that the particulars shown are by way of example and for purposes of illustrative discussion of embodiments of the invention. In this regard, the description taken with the drawings makes apparent to those skilled in the art how embodiments of the invention may be practiced.

FIGS. 1A-G present characterization of Au NPs and PPAu NPs. FIG. 1A (in the inset designated as “1”) and 1B (in the inset designated as “1”) are cryo-TEM images of Au NPs and PPAu NPs, respectively (Bar indicates 200 nm); FIG. 1A (in the inset designated as “2”) and 1B (in the inset designated as “2”) are magnified cryo-TEM images of Au NPs and PPAu NPs, respectively; FIGS. 1C and 1D are size distribution histograms of naked Au NPs and PPAu NPs, respectively; FIG. 1E shows evolution of the ζ-potential versus the number of layers on the Au NPs. FIG. 1F UV-visible spectra of naked Au and PPAu NPs in Tris-HCl and DDW. FIG. 1G shows UV-visible spectra of naked Au NPs and coated Au NPs with different layers. (NPs: nanoparticles; DDW: double distilled water; PPAu: PAA-PEI-Au; PAA: poly(acrylic acid); PEI: polyethylenimine. Cryo-TEM: cryogenic transmission electron microscopy. UV: ultra violet).

FIGS. 2A-D present (in FIG. 2A-C) Agarose gel (1%) electrophoresis of: Naked Au NPs with: 0, 2.5, 5, 10, 20 μg/mL SDFα, (lanes 1-5, respectively). Lane 6: SDFα without NPs (FIG. 2A); PEI-Au NPs with: 0, 2.5, 5, 10, 20, and 30 μg/mL SDFα, (lanes 1-6, respectively) (FIG. 2B); PPAu NPs with: 0, 2.5, 5, 10, 20, and 30 μg/mL SDFα, (lanes 1-6, respectively) (FIG. 2C), and (in FIG. 2D) cryo-TEM images of PPAu-SDFα complexes: (1) 20 μg/mL SDFα; (2) 30 μg/mL SDFα. Insets (1.1), (2.1), and (2.2) in FIG. 2D are magnifications of the rectangular domains in (1)&(2). White arrows point at the SDFα matrix.

FIG. 3 shows SDS-PAGE analysis showing SDFα harvesting by PPAu NPs: (1) FBS; (2) SDFα; (3) SDFα+FBS (4) supernatant; (5) SDFα harvested by PPAu NPs (SDF: Stromal Derived Factor; SDS: sodium dodecyl sulfate. FBS: fetal bovine serum).

FIG. 4 shows mean normalized fluorescent intensity values of: FITC-labeled peptide (PK (FK) 5P) in FBS solution (left); Free FITC-labeled peptide (PK(FK)5P) in supernatant after PPAu separation (middle); Free FITC-labeled peptide (PK(FK)5P) in supernatant after naked Au NPs separation (right) (three replicate analyses and standard deviation are shown) (FITC: Fluorescein isothiocyanate).

FIG. 5 shows SDS-PAGE analysis showing PDGF B partial harvesting from FBS by PPAu nanoparticles in (1) FBS; (2) PDGF B; (3) FBS+ PDGF B; (4) supernatant; (5) PDGF B harvested by PPAu nanoparticles (PDGF: Platelet Derived Growth Factor).

FIG. 6 shows mBCA readings of control solution of trypsin (0.023±0.001) and free trypsin in a solution after its incubation with PPAu NPs (0.021±0.002) (mBCA: micro bicinchoninic acid).

FIG. 7 shows SDS-PAGE results showing the harvesting of SDFα from FBS by PPAu nanoparticles in the presence of a positively charged protein PDGF B: (1) FBS +PDGF B +SDFα; (2) Supernatant; (3) SDFα +PDGF B harvested by PPAu nanoparticles.

FIG. 8 shows ELISA reading of control solution of FBS+ SDFα (3.88±0.198 ng/mL, 1.88±0.04 ng/mL) and SDFα concentration in supernatant (3.13±0.076 ng/mL, 1.60±0.169 ng/mL) and on PPAu NPs (0.75±0.123 ng/mL, 0.28±0.16) in Exp. 1 and Exp. 2, respectively, as described in the Examples section. (ELISA: enzyme-linked immunosorbent assay).

FIGS. 9A-C present Atomic Force Microscope (AFM) measurements demonstrating a significant difference between naked PPAu nanoparticles (FIG. 9A) and complexes of SDFα (0.02 mg/mL an 0.03 mg/mL) and PPAu nanoparticles (FIG. 9B, 9C respectively).

FIG. 10 presents a schematic description of the layer-by-layer (LbL) strategy to functionalize Au nanoparticles according to some embodiments of the invention.

FIG. 11 presents a schematic illustration of harvesting SDFα from FBS by PPAu NPs according to some embodiments of the invention. (I) LMW biomarker incubated with FBS; (II) PPAu NPs added to FBS and LMW biomarker; (III) LMW biomarker adsorbed on PPAu NPs; (IV) PPAu NPs separated by centrifugation from the dispersion for analysis. (LMW: low molecular weight)

FIGS. 12A-B present schematic illustration of BSA-Au NPs complexes formation: (a) Naked Au NPs aggregations in Tris-HCl (20 mM), (b) formation of BSA layer on Au NP's surface (c) formation of multiple BSA layers on Au NP's surface (FIG. 12A); and a schematic illustration of the formation of protein-Au NPs complexes: (a) Naked Au NPs' aggregates in Tris-HCl (20 mM); (b) protein-Au NPs aggregates: (1) cationic trypsin or LL-37 peptide at low concentration, (2) PK(FK)5PK peptide at low concentration; (c) typical morphologies of protein-Au NPs aggregates obtained at higher protein concentrations: (1) cationic trypsin or LL-37 peptide (amorphous aggregates), (2) PK(FK)5PK peptide (fibrillary aggregates) (FIG. 12B).

FIGS. 13A-D present changes in the UV-visible spectra of: naked Au NPs upon the addition of different concentrations of BSA (FIG. 13A): addition of BSA leads to spectral red shift; naked Au NPs upon the addition of cationic trypsin at different concentrations. Addition of cationic trypsin leads to spectral blue shift (FIG. 13B); naked Au NPs related to different concentrations of the cationic peptide LL-37 (blue shift effect) (FIG. 13C); and naked Au NPs related to different concentrations of the PK(FK)5PK peptide (blue shift effect) (FIG. 13D).

FIGS. 14A-D present Cryo-TEM images of Au-protein interaction: Au NPs +BSA (13.8 μM) (FIG. 14A); naked Au NPs+Cationic trypisn (6 μM) (FIG. 14B); Au NPs+LL-37 (222.5 μM) (FIG. 14C); Au NPs+PK(FK)5PK (436.8 μM) (FIG. 14D). The 4 different sub-images in each FIG. 14A-D were sampled from different grid zones.

FIGS. 15A-D present the position of plasmon peak versus protein concentration for naked Au (▪) and PPAu NPs (•) with BSA (FIG. 15A); cationic trypsin (FIG. 15B); LL37 peptide (FIG. 15C); and PK(FK)5PK peptide (FIG. 15D).

FIGS. 16A-D present the UV/visible spectra of PPAu NPs upon the addition of: BSA at different concentrations (FIG. 16A); cationic trypsin at different concentrations (FIG. 16B); cationic LL-37 peptide at different concentrations (FIG. 16C); PK(FK)5PK peptide at different concentrations (FIG. 16D).

FIGS. 17A-D present Cryo-TEM images of PPAu-protein interaction: PPAu NPs+BSA (13.8 μM) (FIG. 17A); PPAu NPs+ Cationic trypisn (6 μM) (FIG. 17B); PPAu NPs+LL-37 (222.5 μM) (FIG. 17B); PPAu NPs+PK(FK)5PK (436.8 μM) (FIG. 17D); The 4 different sub-images in each FIGS. A-D were sampled from different grid zones.

FIG. 18 presents schematic illustration of protein-Au NPs complexes formation process: (a) PPAu NPs in Tris-HCl (20 mM); (b.1 and b.2) LL-37 −PPAu NPs aggregates: (1) LL-37 low concentration, (2) high concentration; (c.1 and c.2) PK(FK)5PK −PPAu NPs complexes formation; (d) PPAu NPs were enclosed within peptide molecules matrix at high concentrations.

FIGS. 19A-B present graphs showing fraction of proteins bound to the NPs as a function of the initial protein concentration, according to the equation described hereinbelow. FIG. 19B further describes the effect of Hill coefficient n on curve shape.

### DETAILED DESCRIPTION OF THE INVENTION

The present invention provides, in one embodiment, a nanostructure being charged comprising at least one gold nanoparticle.

In one embodiment, the nanostructure being charged comprises at least one gold nanoparticle wherein the at least one gold nanoparticle bears on at least portion thereof a positively charged polymer. In one embodiment, the gold nanoparticle is negatively charged.

In another embodiment, the present invention provides, nanostructure being charged comprising at least one gold nanoparticle, wherein the at least one gold nanoparticle: (a) bears on at least portion thereof a positively charged polymer, wherein the positively charged polymer bears on at least portion thereof a negatively charged polymer; and (b) has an averaged diameter of between 5 and 20 nm, and wherein the nanostructure has a hydrodynamic diameter of less than 100 nm, e.g., less than 25 nm. In another embodiment, the negatively charged polymer bears on at least portion thereof a positively charged polymer.

Hereinthroughout, in some embodiments of the nanostructure, by “diameter” it is meant to refer to “at least one dimension thereof” and by “average diameter” it is meant to refer to an average dimensions of one or more nanostructures.

In some embodiments, the term “average” refers to median.

In another embodiment, the present invention provides nanostructure being negatively charged comprising at least one gold nanoparticle, wherein the at least one gold nanoparticle: (a) bears on at least portion thereof a positively charged polymer wherein the positively charged polymer bears on at least portion thereof a negatively charged polymer; and (b) has an averaged diameter of between 5 and 20 nm, and wherein the nanostructure has a hydrodynamic diameter of less than 100 nm, e.g., less than 25 nm.

In another embodiment, the present invention provides nanostructure being positively charged comprising at least one gold nanoparticle, wherein the at least one gold nanoparticle: (a) bears on at least portion thereof a positively charged polymer wherein the positively charged polymer bears on at least portion thereof a negatively charged polymer; the negatively charged polymer bears on at least portion thereof a positively charged polymer and (b) has an averaged diameter of between 5 and 20 nm, and wherein the nanostructure has a hydrodynamic diameter of less than 100 nm, e.g., less than 25 nm.

The present invention provides, in another embodiment, nanostructure being negatively charged comprising at least one gold nanoparticle, wherein the at least one gold nanoparticle: (a) bears on at least portion thereof a positively charged polymer selected from, without limitation, polyethylenamine (PEI), cationic polyallylamine, poly-L-lysine, poly(allylamine hydrochloride), and poly(diallyldimethylammonium chloride), wherein the positively charged polymer bears on at least portion thereof a negatively charged polymer selected from, without limitation, poly(acrylic acid) (PAA), polystyrenesulfonate, polyvinyl sulfate and polyvinylsulfonic acid; and (b) has an averaged diameter of between 5 and 20 nm, and wherein the nanostructure has a hydrodynamic diameter of less than 100 nm, e.g., less than 25 nm.

In another embodiment, there is provided here positively charged nanostructure comprising at least one gold nanoparticle, wherein the at least one gold nanoparticle: (a) bears on at least portion thereof a positively charged polymer selected from, without limitation, polyethylenamine (PEI), cationic polyallylamine, poly-L-lysine, poly(allylamine hydrochloride), and poly(diallyldimethylammonium chloride), wherein the positively charged polymer bears on at least portion thereof a negatively charged polymer selected from, without limitation, poly(acrylic acid) (PAA), polystyrenesulfonate, polyvinyl sulfate and polyvinylsulfonic acid, wherein the negatively charged polymer bears on at least portion thereof a positively charged polymer selected from, without limitation, polyethylenamine (PEI), cationic polyallylamine, poly-L-lysine, poly(allylamine hydrochloride), and poly(diallyldimethylammonium chloride); and (b) has an averaged diameter of between 5 and 20 nm, and wherein the nanostructure has a hydrodynamic diameter of less than 100 nm, e.g., less than 25 nm.

In another embodiment, there is provided here a charged nanostructure comprising at least one gold nanoparticle, wherein the at least one gold nanoparticle: bears on at least portion thereof a positively charged polymer selected from, without limitation, polyethylenamine (PEI), cationic polyallylamine, poly-l-lysine, poly(allylamine hydrochloride), and poly(diallyldimethylammonium chloride), wherein the positively charged polymer bears on at least portion thereof a negatively charged polymer selected from, without limitation, poly(acrylic acid) (PAA), polystyrenesulfonate, polyvinyl sulfate and polyvinylsulfonic acid; and (b) has an averaged diameter of between 5 and 20 nm, and wherein the nanostructure has a hydrodynamic diameter of less than 100 nm, e.g., less than 25 nm.

In another embodiment, provided here nanostructure being a negatively charged comprising at least one gold nanoparticle, wherein the at least one gold nanoparticle: (a) bears on at least portion thereof a positively charged polymer selected from, without limitation, polyethylenamine (PEI), cationic polyallylamine, poly-L-lysine, poly(allylamine hydrochloride), and poly(diallyldimethylammonium chloride), wherein the positively charged polymer bears on at least portion thereof a negatively charged polymer selected from, without limitation, poly(acrylic acid) (PAA), polystyrenesulfonate, polyvinyl sulfate and polyvinylsulfonic acid; and (b) has an averaged diameter of 5 nm, or 6 nm, or 7 nm, or 8 nm, or 9 nm, or 10 nm, or 11 nm, or 12 nm, or 13 nm, or 14 nm, or 15 nm, or 16 nm, or 17 nm, or 18 nm, or 19 nm, or 20 nm, or 21 nm, or 22 nm, or 23 nm, or 24 nm, or 25 nm, or 50 nm or 100 nm, or any value therebetween. In some embodiments the nanostructure has a hydrodynamic diameter of less than e.g., 25 nm, 24 nm, 23 nm, 22 nm, or 21 nm.

In another embodiment, there is provided nanostructure being negatively charged comprising one gold nanoparticle, wherein the gold nanoparticle: (a) bears on at least portion thereof a positively charged polymer selected from, without limitation, polyethylenamine (PEI), cationic polyallylamine, poly-L-lysine, poly(allylamine hydrochloride), and poly(diallyldimethylammonium chloride), wherein the positively charged polymer bears on at least portion thereof a negatively charged polymer selected from, without limitation, poly(acrylic acid) (PAA), polystyrenesulfonate, polyvinyl sulfate and polyvinylsulfonic acid; and (b) has an averaged diameter of between 5 and 20 nm, and wherein the nanostructure has a hydrodynamic diameter of less than 100 nm, e.g., less than 25 nm.

As used herein throughout, the term “polymer” describes an organic substance composed of a plurality of repeating structural units (backbone units) covalently connected to one another. In another embodiment, the polymer is branched. In another embodiment, the polymer is not branched.

Derivatives and analogs of the polymers described herein are also encompassed by the present invention.

The term “derivative” describes a compound which has been subjected to a chemical modification while maintaining its main structural features. Such chemical modifications can include, for example, replacement of one or more substituents and/or one or more functional moieties, oxidation, reduction, and the like.

Hereinthroughout, the terms “nanoparticle”, “nanostructure”, “nano”, “nanosized”, and any grammatical derivative thereof, which are used herein interchangeably, describe a particle featuring a size of at least one dimension thereof (e.g., diameter, length) that ranges from about 1 nanometer to 1000 nanometers. Hereinthroughout NP(s) designates nanoparticle(s). In another embodiments the gold nanoparticle(s) are coated with a capping agent (e.g., citrate).

In another embodiment, the size of the particles described herein represents an average size of a plurality of nanoparticle composites or nanoparticles.

In another embodiment, the charge of the polymers (e.g., negatively charged polymer and/or positively charged polymer) is dictated by the isoelectric point. The isoelectric point (pI) is the pH at which a particular molecule carries no net electrical charge. In another embodiment, the “charge” refers to the polymer in a medium having pH of about 7.

In some embodiments, the term “isoelectric point” refers to the point at which a molecule or compound, which can exist in forms bearing either negative and/or positive charges, is electrically balanced, such that the net charge on the molecule or compound is zero.

The term “portion” should be construed as meaning part or all of the surface of one or more nanoparticles. In another embodiment, the term “portion” refers to the upper surface. In another embodiment, the positively charged polymer and/or the negatively charged polymer have a form of a layer or coating and the term “portion” refers to the upper surface of the layer or coating.

In another embodiment, by “bear” it is meant to refer to covalent interactions. In another embodiment, by “bear” it is meant to refer to non-covalent interactions. By “non-covalent interactions” it is meant to refer to binding, by any non-covalent interaction, to another molecule, ion, complex or substance. The non-covalent interactions include, but are not limited to, ionotropic interaction, complexation interaction, electrostatic interactions, hydrogen bonds, receptor-substrate interactions, or any other non-covalent crosslinking and combinations thereof.

In some embodiments, the disclosed nanostructure(s) has a spherical shape. In some embodiments, the disclosed nanostructure(s) has an elongated shape such as wire, tube, rod, bipod, tripod and tetrapod.

In another embodiment, the positively charged polymer is polyethylenamine (PEI). In another embodiment, the positively charged polymer is cationic polyallylamine, poly-L-lysine. In another embodiment, the positively charged polymer is poly(allylamine hydrochloride).

In another embodiment, the negatively charged polymer is poly(acrylic acid) (PAA). In another embodiment, the negatively charged polymer is polystyrenesulfonate. In another embodiment, the negatively charged polymer is polyvinylsulfonic acid. In another embodiment, the negatively charged polymer is polyvinyl sulfate.

Hence, in another embodiment, there is provided a nanostructure being negatively charged (denoted as: “PAA-PEI-Au”) comprising at least one gold nanoparticle, wherein the at least one gold nanoparticle: (a) bears on at least portion thereof polyethylenamine (PEI), wherein the positively charged polymer bears on at least portion thereof poly(acrylic acid) (PAA), and (b) has an averaged diameter of between 5 and 20 nm, and wherein the nanostructure has a hydrodynamic diameter of less than 100 (e.g., less than 25 nm).

In another embodiment, the positively charged polymer has an average molecular weight (MW) that ranges from 6 kDa to 50 kDa. In another embodiment, the positively charged polymer has an average molecular weight (MW) that ranges from 6 kDa to 30 kDa. In another embodiment, the positively charged polymer has an average molecular weight (MW) that ranges from 6 kDa to 15 kDa. In another embodiment, the positively charged polymer has an average MW of 6 kDa. In another embodiment, the positively charged polymer has an average MW of about 7 kDa. In another embodiment, the positively charged polymer has an average MW of about 8 kDa. In another embodiment, the positively charged polymer has an average MW of about 9 kDa. In another embodiment, the positively charged polymer has an average MW of about 10 kDa. In another embodiment, the positively charged polymer has an average MW of about 11 kDa. In another embodiment, the positively charged polymer has an average MW of about 12 kDa. In another embodiment, the positively charged polymer has an average MW of about 13 kDa. In another embodiment, the positively charged polymer has an average MW of about 14 kDa. In another embodiment, the positively charged polymer has an average MW of about 15 kDa.

Herein, the term “average molecular weight” is meant to refer to weight average molecular weight. As used herein, the term “weight average molecular weight” generally refers to a molecular weight measurement that depends on the contributions of polymer molecules according to their sizes.

In another embodiment, the negatively charged polymer has an average molecular weight (MW) that ranges from 8 kDa to 15 kDa.

In another embodiment, the negatively charged polymer has an average MW of about 8 kDa. In another embodiment, the negatively charged polymer has an average MW of about 9 kDa. In another embodiment, the negatively charged polymer has an average MW of about 10 kDa. In another embodiment, the negatively charged polymer has an average MW of about 11 kDa. In another embodiment, the negatively charged polymer has an average MW of about 12 kDa. In another embodiment, the negatively charged polymer has an average MW of about 13 kDa. In another embodiment, the negatively charged polymer has an average MW of about 14 kDa. In another embodiment, the negatively charged polymer has an average MW of about 15 kDa.

In another embodiment, the disclosed nanostructure is characterized by a high affinity to a positively charged low molecular weight (LMW) molecules. In another embodiment, the disclosed nanostructure is characterized by a high affinity to a negatively charged low molecular weight (LMW) molecules.

The term “high affinity” is meant to refer specifically binding a target molecule (e.g., LMW molecule) with an affinity higher than e.g., 10^{˜6} M. Specific binding can be detected by various assays as long as the same assay conditions are used to quantify binding to the target versus control, and as such is stable under physiological (e.g., in vivo) conditions.

In another embodiment, the disclosed nanostructure being negatively charged has high affinity to positively charged LMW molecule. In another embodiment, the disclosed nanostructure being positively charged has high affinity to negatively charged LMW molecule.

Thus, the binding affinity (e.g., to binding domain of the LMW molecule) is higher than (i.e., at least) about, 10⁻⁴ M, 10⁻⁵ M, 10⁻⁶ M, 10⁻⁷ M, 10⁻⁸ M, 10⁻⁹ M, or 10⁻¹⁰ M. In addition, binding of the high affinity molecule does not interfere with proteolysis by e.g., blood-plasma proteases or high molecular weight proteins.

In another embodiment, the LMW molecules are peptides. In another embodiment, the LMW molecules are proteins. In another embodiment, the LMW molecules are labeled (e.g., fluorescently labeled e.g., by FITC).

In another embodiment, the LMW molecules are activated platelets. In another embodiment, the LMW molecules are platelet-derived microparticles. In another embodiment, the LMW molecules are apoptotic bodies. In another embodiment, the LMW molecules are antibodies (e.g., IgG). In another embodiment, the LMW molecules are biomarkers as described hereinbelow.

In another embodiment, the LMW molecules have a MW value of less than 50 kDa, or in another embodiment, less than 40 kDa, or in another embodiment, less than 30 kDa, or in another embodiment, less than 20 kDa, or in another embodiment, less than 15 kDa, or in another embodiment, less than 14 kDa, or in another embodiment, less than 13 kDa, or in another embodiment, less than 12 kDa, or in another embodiment, less than 11 kDa, or in another embodiment, less than 10 kDa, or in another embodiment, less than 9 kDa, or in another embodiment, less than 8 kDa, or in another embodiment, less than 7 kDa, or in another embodiment, less than 6 kDa, or in another embodiment, less than 5 kDa, or in another embodiment, less than 4 kDa, or in another embodiment, less than 3 kDa, or in another embodiment, less than 2 kDa, or in another embodiment, less than 1 kDa.

In another embodiment, the LMW molecules further refer to a group of proteins in which the MW varies within less than 30%, less than 20%, or less than 10%.

In another embodiment, the LMW molecules are Stromal Derived Factor-alpha (SDF-alpha). In another embodiment, the LMW molecules are Platelet Derived Growth Factor B.

In another embodiment, the disclosed negatively charged nanostructure is characterized by a zeta-potential of at least |−30| mV in aqueous dispersion. In another embodiment, the disclosed nanostructure is characterized by a zeta-potential of at least |−35| mV in aqueous dispersion. In another embodiment, the disclosed nanostructure is characterized by a zeta-potential of at least |−40| mV in aqueous dispersion. In another embodiment, the disclosed nanostructure is characterized by a zeta-potential of at least |−45| mV in aqueous dispersion. In another embodiment, the disclosed nanostructure is characterized by a zeta-potential of at least |−50| mV in aqueous dispersion.

In another embodiment, the disclosed positively charged nanostructure is characterized by a zeta-potential of at least |+30| mV in aqueous dispersion. In another embodiment, the disclosed nanostructure is characterized by a zeta-potential of at least |+35| mV in aqueous dispersion. In another embodiment, the disclosed nanostructure is characterized by a zeta-potential of at least |+40| mV in aqueous dispersion. In another embodiment, the disclosed nanostructure is characterized by a zeta-potential of at least |+45| mV in aqueous dispersion. In another embodiment, the disclosed nanostructure is characterized by a zeta-potential of at least |+50| mV in aqueous dispersion.

In another embodiment, there is provided a composition comprising a plurality of the disclosed nanostructures. In another embodiment, the composition is in the form of stable dispersion. The terms “stable dispersion”, or “stable colloidal dispersion” refer to a dispersion having discrete nanostructures which is stable, i.e., the disperse phase of the dispersion substantially does not flocculate, for a period of at least about 30 minutes, 1 h, 2 h, 3 h 4 h, 5 h, 6 h, 7 h, 8 h, 9 h 10 h, 15 h, or 20 h.

In another embodiment, the composition is characterized by at least 90% of the plurality of the nanostructures having an averaged diameter that varies within less than ±20%. In another embodiment, at least 80%, at least 85%, at least 90%, or at least 95%, of the plurality of the nanostructures having an averaged diameter that varies within less than ±20%. In another embodiment, the composition is characterized by at least 90% of the plurality of the nanostructures having an averaged diameter that varies within less than ±5%, ±10%, ±15%, ±20%, or ±25%.

In another embodiment, the disclosed composition comprises an active agent (charged active agent) on a surface thereof. In some embodiments, this target agent may be released in a target cell or tissue.

In some embodiments, the disclosed composition is for use in diagnosis and/or detection of medical disorder or disease. In another embodiment, the disclosed composition is for use in diagnosis and/or detection of medical disorder or disease, the medical disorder or disease being characterized by occurrence of excessive charged low molecular weight (LMW) molecules. In another embodiment, the disease is cancer. In another embodiment, the disease is inflammation. In another embodiment, the detection is in a sample of biological cells. In another embodiment, the detection is in vitro. In another embodiment, the detection is ex vitro. In another embodiment, the detection is in vivo. In another embodiment, the detection is for clinical imaging. In another embodiment, the detection is used to characterize the intrinsic apoptotic load within a tumor. In another embodiment, the detection is used to the level of aggressiveness of a tumor. In another embodiment, is used to detect metastases.

In another embodiment, the disclosed composition is for use in clinical applications, e.g., as drug delivery vehicle, as well as in gene therapy and for diagnostic imaging.

In the context of drug delivery, the composition, according to some embodiments of the present invention, can be used for delivering a variety of bioactive and therapeutic agents which are useful in the treatment of a variety of medical conditions, including, without limitation, inflammation, infection, tumor suppression, bone tissue formation, tissue proliferation, metabolite and endocrine regulation, pain relief, and the likes.

The terms “cancer” and “tumor”, are used interchangeably herein to describe a class of diseases in which a group of cells display uncontrolled growth (division beyond the normal limits). The term “cancer” encompasses malignant and benign tumors as well as disease conditions evolving from primary or secondary tumors. The term “malignant tumor” describes a tumor which is not self-limited in its growth, is capable of invading into adjacent tissues, and may be capable of spreading to distant tissues (metastasizing). The term “benign tumor” describes a tumor which is non-malignant (i.e. does not grow in an unlimited, aggressive manner, does not invade surrounding tissues, and does not metastasize). The term “primary tumor” describes a tumor that is at the original site where it first arose. The term “secondary tumor” describes a tumor that has spread from its original (primary) site of growth to another site, close to or distant from the primary site. Metastases or metastatic cancer is a cancer that has spread from the part of the body where it started (the primary site) to other parts of the body.

In another embodiment, the composition further comprises a physiologically acceptable carrier. In another embodiment, the phrases “physiologically acceptable carrier” refers to a carrier or a diluent that does not cause significant irritation to an organism and does not abrogate the biological activity and properties of the administered compound. An adjuvant is included under these phrases.

In another embodiment, the composition further comprises a radiolabel. In another embodiment, the composition further comprises an X-ray contrast agent. In another embodiment, the composition further comprises a magnetic resonance imaging (MM) contrast agent. In another embodiment, the composition further comprises a fluorescent label. In another embodiment, the disclosed composition may be detected by a method selected from fluorescent microscope, a flow-cytometric equipment, MRI contrast, X-ray or computerized tomography (CT) contrast.

In another embodiment, the detection is used to characterize the intrinsic apoptotic load within a tumor, the level of aggressiveness of a tumor, or to detect metastases.

In another embodiment, there is provided a method for the detection of positively charged LMW molecules in a sample. In another embodiments, the method comprises:

(i) contacting the sample with the composition of some embodiments disclosed herein comprising the negatively charged nanostructure under conditions enabling binding of the nanostructures to charged LMW molecules; and

(ii) detecting bound nanostructures to the LMW molecules, thereby indicating the presence of charged LMW molecules.

In another embodiment, there is provided a method for the detection of negatively charged LMW molecules in a sample. In another embodiments, the method comprises:

(i) contacting the sample with the composition of some embodiments disclosed herein comprising the positively charged nanostructure under conditions enabling binding of the nanostructures to charged LMW molecules; and

(ii) detecting bound nanostructures to the LMW molecules, thereby indicating the presence of charged LMW molecules.

In another embodiments, the sample is selected from blood serum and blood plasma.

In another embodiment, there is provided a method for the detection of a physiological disorder or disease characterized by the presence charged LMW molecules, the method comprising:

(i) administering a composition according to some embodiments disclosed herein to a patient in need thereof; and

(ii) imaging the patient using an appropriate imaging technique as described herein.

In another embodiment, by “conditions enabling binding of the nanostructures to charged LMW molecules” it is meant to refer to physiological conditions. In another embodiment, by “conditions enabling binding of the nanostructures to charged LMW molecules” it is meant to refer to pH of e.g., 5, 6, 7, or 8, including any value therebetween. In another embodiment, by “conditions enabling binding of the nanostructures to charged LMW molecules” it is meant to refer to room temperature, (i.e. 15° C. to 30° C.). In another embodiment, by “conditions enabling binding of the nanostructures to charged LMW molecules” it is meant to refer to positively charged LMW molecules. In another embodiment, by “conditions enabling binding of the nanostructures to charged LMW molecules” it is meant to refer to negatively charged LMW molecules.

The composition also comprises, in some embodiments, a preservative, such as benzalkonium chloride and thimerosal and the like; chelating agents, such as edetate sodium and others; buffers such as phosphate, citrate and acetate; tonicity agents such as sodium chloride, potassium chloride, glycerin, mannitol and others; antioxidants such as ascorbic acid, acetylcystine, sodium metabisulfote and others; aromatic agents; viscosity adjustors, such as polymers, including cellulose and derivatives thereof; and polyvinyl alcohol and acid and bases to adjust the pH of these aqueous compositions as needed. The composition also comprises, in some embodiments, local anesthetics or other actives. The disclosed compositions can be used, in some embodiments, as sprays, mists, drops, and the like.

In some embodiments, the pharmaceutical composition is formulated as a pharmaceutically acceptable injectable matrix.

In addition, the compositions of the invention may further comprise binders (e.g. acacia, cornstarch, gelatin, carbomer, ethyl cellulose, guar gum, hydroxypropyl cellulose, hydroxypropyl methyl cellulose, povidone), disintegrating agents (e.g. cornstarch, potato starch, alginic acid, silicon dioxide, croscarmelose sodium, crospovidone, guar gum, sodium starch glycolate), buffers (e.g., Tris-HCI., acetate, phosphate) of various pH and ionic strength, additives such as albumin or gelatin to prevent absorption to surfaces, detergents (e.g., Tween 20, Tween 80, Pluronic F68, bile acid salts), protease inhibitors, surfactants (e.g. sodium lauryl sulfate), permeation enhancers, solubilizing agents (e.g., glycerol, polyethylene glycerol), antioxidants (e.g., ascorbic acid, sodium metabisulfite, butylated hydroxyanisole), stabilizers (e.g., hydroxypropyl cellulose, hyroxypropylmethyl cellulose), viscosity increasing agents (e.g., carbomer, colloidal silicon dioxide, ethyl cellulose, guar gum), sweeteners (e.g., aspartame, citric acid), preservatives (e.g., Thimerosal, benzyl alcohol, parabens), lubricants (e.g., stearic acid, magnesium stearate, polyethylene glycol, sodium lauryl sulfate), flow-aids (e.g., colloidal silicon dioxide), plasticizers (e.g. diethyl phthalate, triethyl citrate), emulsifiers (e.g., carbomer, hydroxypropyl cellulose, sodium lauryl sulfate), polymer coatings (e.g., poloxamers or poloxamines), coating and film forming agents (e.g., ethyl cellulose, acrylates, polymethacrylates) and/or adjuvants.

Typical components of carriers for syrups, elixirs, emulsions and suspensions include ethanol, glycerol, propylene glycol, polyethylene glycol, liquid sucrose, sorbitol and water. For a suspension, typical suspending agents include methyl cellulose, sodium carboxymethyl cellulose, cellulose (e.g. Avicel™, RC-591), tragacanth and sodium alginate; typical wetting agents include lecithin and polyethylene oxide sorbitan (e.g. polysorbate 80). Typical preservatives include methyl paraben and sodium benzoate. In another embodiment, peroral liquid compositions also contain one or more components such as sweeteners, flavoring agents and colorants disclosed above.

In another embodiment, compositions of the present invention are presented in a pack or dispenser device, such as an U.S. Food and Drug Administration (FDA) approved kit, which contain one or more unit dosage forms containing the active ingredient. In one embodiment, the pack, for example, comprises metal or plastic foil, such as a blister pack. In one embodiment, the pack or dispenser device is accompanied by instructions for administration. In one embodiment, the pack or dispenser is accommodated by a notice associated with the container in a form prescribed by a governmental agency regulating the manufacture, use or sale of pharmaceuticals, which notice is reflective of approval by the agency of the form of the compositions or human or veterinary administration. Such notice, in one embodiment, is labeling approved by the FDA for prescription drugs or of an approved product insert.

The Synthesis

In one embodiment, there is provided a process for synthesis of the herein disclosed nanostructures in one or more embodiments thereof, for example, nanostructures being negatively charged comprising a gold nanoparticle, wherein the gold nanoparticle bears on at least portion thereof a positively charged polymer and the positively charged polymer bears on at least portion thereof a negatively charged polymer, the process comprises the steps of:

adding gold nanoparticles into dispersion comprising positively charged polymer in water, thereby forming nanostructures being positively charged, comprising at least one gold nanoparticle, wherein the gold nanoparticle bears the positively charged polymer;

centrifuge the dispersion, thereby purifying the nanostructures;

re-dispersing the nanostructures in water; and, in some embodiments, the synthesis comprises a step of:

adding the re-dispersed nanostructure to a dispersion comprising negatively charged polymer, thereby forming nanostructures being negatively charged comprising a gold nanoparticle, wherein the gold nanoparticle bears on at least portion thereof a positively charged polymer and the positively charged polymer bears on at least portion thereof a negatively charged polymer.

In another embodiment, the gold nanoparticles are prepared while using a capping agent. In another embodiment, the capping agent is citrate. In another embodiment, the gold nanoparticles are citrate-capped gold nanoparticle.

In another embodiment, the step of adding gold nanoparticles into dispersion comprising positively charged polymer in water is performed while stirring. In another embodiment, the step of adding gold nanoparticles into dispersion comprising positively charged polymer in water is followed by a step of stirring the dispersion. In another embodiment, the step of adding gold nanoparticles into dispersion comprising positively charged polymer in water is performed dropwise.

In another embodiment, the step of adding gold nanoparticles into dispersion comprising positively charged polymer in water is followed by a step of stirring the dispersion for at least e.g., 1 h, 2 h, 3 h, 4 h, 5 h, 6 h, 7 h, 8 h, 9 h, 10 h, 11 h, 12 h, 13 h, 14 h, 15 h, 16 h, 17 h, or 18 h.

In another embodiment, the nanostructures being negatively charged are centrifuge so as to purify thereof.

Herein, the term “centrifuge” defines a machine using centrifugal force for separating substances of different densities. The centrifugation may be performed at 100 rpm, 200 rpm, 300 rpm, 400 rpm, 500 rpm, 600 rpm, 700 rpm, 800 rpm, 900 rpm, 1000 rpm, 2000 rpm, 3000 rpm, 4000 rpm, 5000 rpm, 6000 rpm, 7000 rpm, 8000 rpm, 9000 rpm, or 10000 rpm, including any value therebetween.

Protein Separation

In some embodiments, the present invention provides a method for separating, purifying and/or characterizing LMW molecules (e.g., proteins) from a mixture comprising: passing the mixture through at least one nanostructure disclosed hereinabove, thereby adsorbing the proteins (e.g., LMW proteins and or a biomarker) on the disclosed nanostructure. In some embodiments, the method comprises assaying the adsorbed proteins, by any method known in the art. In some embodiments, the method comprises assaying the remaining proteins in the mixture, by any method known in the art. In some embodiments, the method comprises separating the adsorbed protein(s) from the nanostructure by any method known in the art (e.g., centrifugation). In some embodiments, the separation is performed while preserving protein function or enzyme activity.

In some embodiments, the molecules (e.g., proteins) in the mixture may be purified according to their isoelectric point (PI). In certain embodiments, this separation utilizes no reducing agents. In various embodiments, the proteins in the mixture may be purified according to their molecular weight. In various embodiments, the proteins in the mixture may be purified both according to their isoelectric points and their molecular weight. In some embodiments the proteins are the charged LMW molecules, as described hereinabove, for example positive charged LMW.

In some embodiments, by “separating, purifying and/or characterizing proteins from a mixture”, it is meant, inter alia, that at least 60%, 70%, 80%, or 90% of the molecules (e.g., proteins) are adsorbed on the nanostructure surface. In some embodiments, by “purified according to their molecular weight” it is meant, inter alia that the molecular weights of the molecules protein adsorbed on the nanostructure vary within less than ±50%, ±40%, ±30%, ±20%, or ±10%, thereby e.g., allowing to remove undesired proteins from the mixture.

In some embodiments, the PI is above 7, above 8, above 9, above 10, above 11, or above 12. In some embodiments, the PI is below 7, below 6, below 5, or below 4.

In some embodiments, the disclosed nanostructure harvests LMW proteins from a medium. In some embodiments, the medium is a biological fluid (e.g. a serum). The term “biomarker”, in the context of the current invention, refers to a molecule (e.g., an antibody) present in a biological sample of a patient, the levels of which in said biological fluid may be indicative of a physiological condition (e.g., a disease).

In some embodiments, the method is performed using a kit.

In some embodiments, the kit is for use in detection of a biomarker. In some embodiments, the kit comprises a medium having affixed thereto one or more of the disclosed nanostructures. In some embodiments, the kit comprises a means for acquiring a quantity of LMW molecules. In some embodiments, the kit is for use in detection of a biomarker in a body fluid sample. In some embodiments, the kit comprises more than one types (for examples, nanostructures having a positive charge and nanostructures having a negative charge of the disclosed nanostructures).

In some embodiments, the kit is in the form of a biosensor. The term “biosensor” may particularly denote any device that may be used for the detection of an analyte comprising LMW biological molecules such as, without limitation, proteins, enzymes, virus, DNA, etc. The biosensor may combine a biological component (for instance capture molecules at a sensor active surface capable of detecting molecules) with a physicochemical or physical detector component (for instance a capacitor having a capacitance which is modifiable by a sensor event, or a layer having a redox potential which is modifiable by a sensor event, or a field effect transistor having a threshold voltage or a channel conductivity which is modifiable by a sensor event).

In some embodiments, the term “protein” refers to any chain of amino acids, regardless of length or post-translational modification. Proteins can exist as monomers or multimers, comprising two or more assembled polypeptide chains, fragments of proteins, polypeptides, oligopeptides, or peptides.

In some embodiments, the term “purified protein or peptide” as used herein, is intended to refer to a composition, isolatable from other components, wherein the protein or peptide is purified to any degree relative to its naturally obtainable state. A purified protein or peptide therefore also refers, in some embodiments, to a protein or peptide, free from the environment in which it may naturally occur. A purified protein or peptide is said to “preserve its activity”, if the biological activity of the protein, such as an enzyme, at a given time, is within about 10% (within the errors of the assay) of the biological activity exhibited by the protein in a mixture.

As used herein, the terms “active protein”, “biologically active protein”, “bioactive protein”, “biologically active protein fragment”, or “bioactive protein fragment” refer to any polypeptide or fragment thereof derived from a mixture according to the teaching of this disclosure that has biological activity, e.g., enzymatic activity, etc. Thus, the term “bioactive protein” refers to a protein having biological activity.

In some embodiments, the body fluid sample is selected from, without being limited thereto, urine, blood, serum, plasma, saliva, lymph, cerebrospinal fluid, cystic fluid, ascites, stool, bile, and any isolatable body fluid.

General

As used herein the term “about” refers to ±10%.

The terms “comprises”, “comprising”, “includes”, “including”, “having” and their conjugates mean “including but not limited to”. The term “consisting of” means “including and limited to”. The term “consisting essentially of” means that the composition, method or structure may include additional ingredients, steps and/or parts, but only if the additional ingredients, steps and/or parts do not materially alter the basic and novel characteristics of the claimed composition, method or structure.

The word “exemplary” is used herein to mean “serving as an example, instance or illustration”. Any embodiment described as “exemplary” is not necessarily to be construed as preferred or advantageous over other embodiments and/or to exclude the incorporation of features from other embodiments.

The word “optionally” is used herein to mean “is provided in some embodiments and not provided in other embodiments”. Any particular embodiment of the invention may include a plurality of “optional” features unless such features conflict.

As used herein, the singular form “a”, “an” and “the” include plural references unless the context clearly dictates otherwise. For example, the term “a compound” or “at least one compound” may include a plurality of compounds, including mixtures thereof.

Throughout this application, various embodiments of this invention may be presented in a range format. It should be understood that the description in range format is merely for convenience and brevity and should not be construed as an inflexible limitation on the scope of the invention. Accordingly, the description of a range should be considered to have specifically disclosed all the possible subranges as well as individual numerical values within that range. For example, description of a range such as from 1 to 6 should be considered to have specifically disclosed subranges such as from 1 to 3, from 1 to 4, from 1 to 5, from 2 to 4, from 2 to 6, from 3 to 6 etc., as well as individual numbers within that range, for example, 1, 2, 3, 4, 5, and 6. This applies regardless of the breadth of the range.

Whenever a numerical range is indicated herein, it is meant to include any cited numeral (fractional or integral) within the indicated range. The phrases “ranging/ranges between” a first indicate number and a second indicate number and “ranging/ranges from” a first indicate number “to” a second indicate number are used herein interchangeably and are meant to include the first and second indicated numbers and all the fractional and integral numerals therebetween.

As used herein the term “method” refers to manners, means, techniques and procedures for accomplishing a given task including, but not limited to, those manners, means, techniques and procedures either known to, or readily developed from known manners, means, techniques and procedures by practitioners of the chemical, pharmacological, biological, biochemical and medical arts.

As used herein, the term “treating” includes abrogating, substantially inhibiting, slowing or reversing the progression of a condition, substantially ameliorating clinical or aesthetical symptoms of a condition or substantially preventing the appearance of clinical or aesthetical symptoms of a condition.

In those instances where a convention analogous to “at least one of A, B, and C, etc.” is used, in general such a construction is intended in the sense one having skill in the art would understand the convention (e.g., “a system having at least one of A, B, and C” would include but not be limited to systems that have A alone, B alone, C alone, A and B together, A and C together, B and C together, and/or A, B, and C together, etc.). In those instances where a convention analogous to “at least one of A, B, or C, etc.” is used, in general such a construction is intended in the sense one having skill in the art would understand the convention (e.g., “a system having at least one of A, B, or C” would include but not be limited to systems that have A alone, B alone, C alone, A and B together, A and C together, B and C together, and/or A, B, and C together, etc.). It will be further understood by those within the art that virtually any disjunctive word and/or phrase presenting two or more alternative terms, whether in the description, claims, or drawings, should be understood to contemplate the possibilities of including one of the terms, either of the terms, or both terms. For example, the phrase “A or B” will be understood to include the possibilities of “A” or “B” or “A and B.”

### EXAMPLES

Reference is now made to the following examples which, together with the above descriptions, illustrate the invention in a non-limiting fashion.

### Example 1

### Synthesis

PPAu Synthesis.

Citrate capped gold nanoparticles were prepared according to a published procedure (Greenberg, A. K. et al. PLoS ONE 2012, 7). Chloroauric acid (HAuCl₄) stock solution (19 mL, 0.25 mM) was boiled with vigorous stirring. Then, 1 mL of sodium citrate (0.5%) was added to the boiled solution. The gold solution gradually forms as the citrate reduces the gold (III) to neutral gold atoms and gold gradually starts to precipitate in the form of sub-nanometer particles. Then the solution was boiled for further 30 min with vigorous stirring. A progressive color change was observed from yellowish toward red. The Au nanoparticles were characterized by cryogenic transmission electron microscopy (cryo-TEM), UV-Visible Spectroscopy (UV-vis), ζ-potential.

PEI-Au was prepared using commercially available PEI (10 kDa). PEI cationic polymer was dissolved in ddH₂O (100 mg/mL) and stirred; previously-prepared citrate-capped gold nanoparticle solution was added dropwise to the polymer solution and the solution was stirred overnight. PEI-Au nanoparticles were purified by high speed centrifugation and re-dispersed in ddH₂O; the process was repeated three times. The PEI-Au nanoparticles were characterized by UV-vis, and ζ-potential.

PAA-PEI-Au (denoted as “PPAu”) was prepared using commercially available PAA (8 kDa). PAA anionic polymer was dissolved in ddH₂O, PEI-Au nanoparticle solution was added dropwise to the polymer solution and the solution was stirred overnight. PAA-PEI-Au nanoparticle were purified by high speed centrifugation and re-dispersed in ddH₂O; the process was repeated three times. The PAA-PEI-Au nanoparticles were characterized by cryo-TEM, UV-vis, ζ-potential as described hereinbelow.

### Example 2

### Characterization

Cryo-TEM.

Au and PPAu solution samples were prepared on a copper grid coated with a perforated lacy carbon 300 mesh (Ted Pella Inc.). A typically 4 μl drop from the solution was applied to the grid and blotted with a filter paper to form a thin liquid film of solution. The blotted sample was immediately plunged into liquid ethane at its freezing point (−183° C.). The procedure was performed automatically in the Plunger (Lieca EM GP). The vitrified specimens were transferred into liquid nitrogen for storage. The samples were studied using a FEI Tecnai 12 G2 TEM, at 120 kV with a Gatan cryo-holder maintained at −180° C., and images were recorded on a slow scan cooled charge-coupled device CCD camera Gatan manufacturer. Images were recorded with the Digital Micrograph software package at low dose conditions to minimize electron beam radiation damage.

ζ-Potential.

The surface charge of the naked Au, PEI-Au, and PAA-PEI-Au nanoparticles was evaluated by their ζ-potential. U-tube cuvette (DTS1060C, Malvern) for ζ-potential measurements by Zetasizer (ZN-NanoSizer, Malvern, England) was used. Each of the naked Au, PEI-Au, and PAA-PEI-Au nanoparticle samples were measured in automatic mode, at 25° C., and the Smoluchowski model was used to calculate the zeta potential. For each sample the ζ-potential value was presented as the average value of three runs.

UV-Vis Spectra.

UV-vis spectra of Au, PEI-Au, and PAA-PEI-Au were recorded using an infinite M200 spectrophotometer (TECAN) equipped with a temperature controller.

### Results

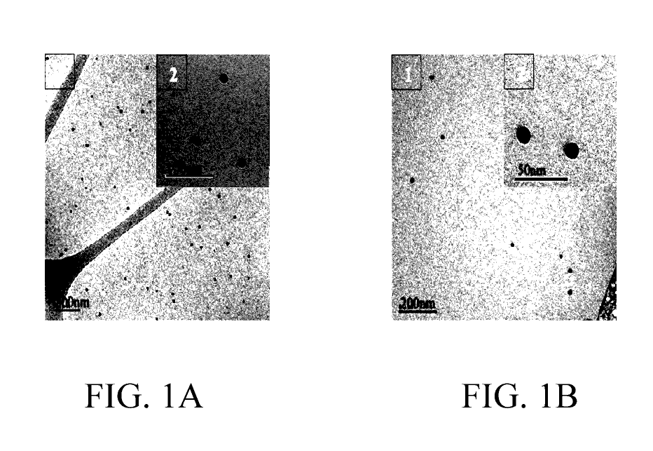

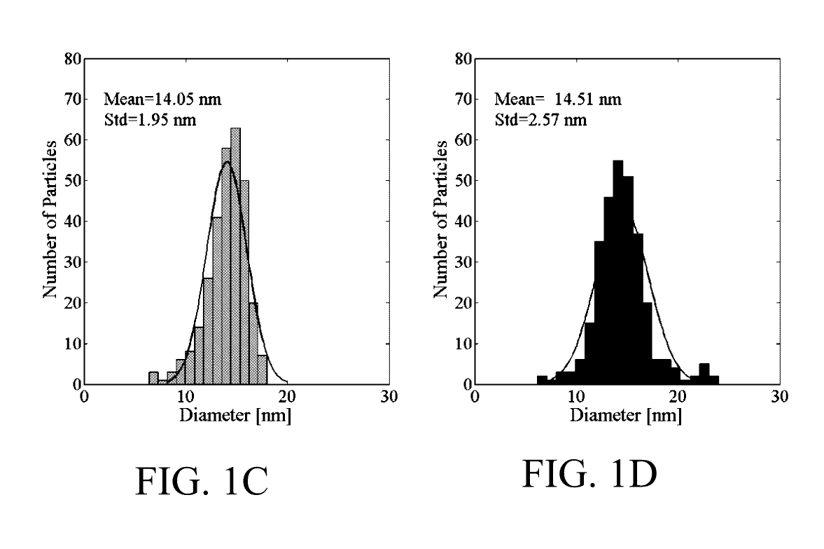

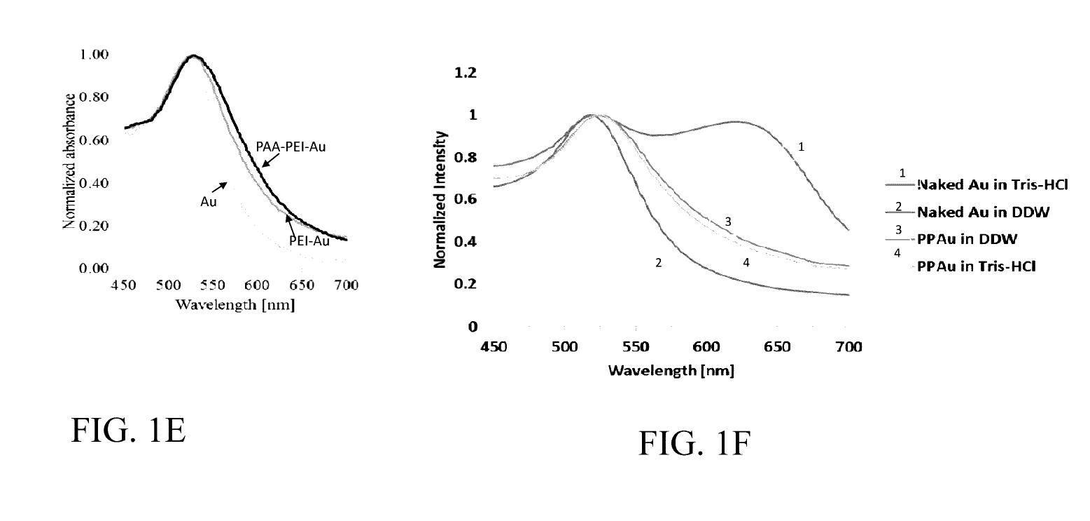

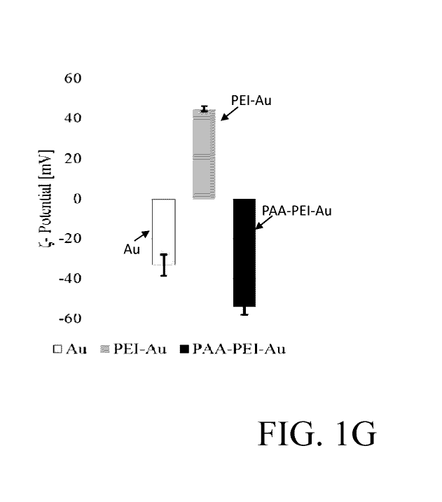

As described hereinabove, first, ˜14 nm diameter, naked, homogeneous and negatively charged Au NPs were synthesized. Next, according to the layer-by-layer (LbL) method the naked NPs were coated with positively charged polyethyleneimine (PEI) polymer (10 kDa), followed by coating with negatively charged PAA (8 kDa). Cryogenic Transmission Electron Microscopy (cryo-TEM) images of naked (FIG. 1A), and coated (FIG. 1B) NPs provided their size distributions as shown in FIG. 1C (mean: 14.05 nm) and FIG. 1D (mean: 14.51 nm), respectively.

The UV-vis spectra revealed that the PPAu remained predominantly disaggregated after each coating as the maximum of the plasmon peak shifted from 520 nm, through 522 nm to 524 nm representing the naked and the consecutively coated NPs, respectively (FIG. 1E). These results were in agreement with mean cryo-TEM diameter readings of ˜14.5 nm after the final layer deposition (FIGS. 1A-D) showing no significant deviation from naked NPs. An aqueous solution of naked Au NPs in DDW showed a narrow and nearly symmetric absorption peak at approximately 518 nm (FIG. 1F). This indicates that naked Au NPs are monodispersed and spherical. Normally, the negatively charged double layer surrounding the naked Au NPs applies repulsive forces that balance the van der Waals attraction forces, prevent NP aggregation and increase stability. Addition of Tris-HCl (20 mM) solution to naked Au NPs' dispersion in DDW reduced the magnitude of their ζ-potential below 30 mV allowing the van der Waals attraction to out-weigh the repulsive forces and induce aggregation. The aggregation is manifested by a red shift and broadening of the UV-vis spectral absorption band (FIG. 1F). On the other hand, addition of Tris-HCl (20 mM) to PPAu NPs' dispersion in DDW did not cause aggregation because of their higher inherent stability (ζ-potential˜−53 mV in DDW). These results were corroborated by the UV-Vis spectral.

The alternation of ζ-potential from −33.18±5.47 mV, to +45.02±1.31 mV, and then to −53.82±4.28 mV, after the coating completion of each layer, confirmed the deposition of each polymer on the nanoparticle surface (FIG. 1G).

### Example 3

### SDFα Adsorption on PPAu

To monitor SDFα (8 kDa, pI 10.3) adsorption on PPAu nanoparticles, the peptide was incubated with constant concentration of PPAu nanoparticles while the peptide's concentration was varied from 0 to 30 μg/mL. The adsorption process was characterized by Agarose gel, and cryo-TEM.

Agarose Gel.

Agarose gel (1%) was used to monitor adsorption of SDFα on PPAu, PEI-Au, and on naked Au NPs. 20 μL of each PPAu and PEI-Au NPs dispersions (5.86×10¹¹ NPs/mL) were incubated with solutions of SDFα at various concentrations (2.5, 5, 10, 20, and 30 μg/mL) in 20 μL of 20 mM Tris-HCl pH=7 for 1 h at room temperature (RT). 20 μL of naked Au NPs dispersions were incubated with SDFα solutions at various concentrations (2.5, 5, 10, and 20 μg/mL in 20 μL of 20 mM Tris-HCl pH=7), and the 6^{th} well was used as a control for SDFα (20 m/mL). Subsequently, 8 μL of loading dye was added to each sample, and the entire mixture was loaded into the gel's wells (40 μL size). A constant voltage (90 V) was applied for 25 min to ensure sufficient separation. The gel was stained by Coomassie Blue for 2 h, followed by water wash (200 mL) until protein bands were clear.

Cryo-TEM.

The same samples used in agarose gel of PPAu, and PPAu+SDFα (20 μg/mL and 30 μg/mL) complexes were prepared on a copper grid coated with a perforated lacy carbon 300 mesh (Ted Pella Inc.). A typically 4 μl drop from the solution was applied to the grid and blotted with a filter paper to form a thin liquid film of solution. The blotted samples were immediately plunged into liquid ethane at its freezing point (−183° C.). The procedure was performed automatically in the Plunger (Lieca EM GP). The vitrified specimens were transferred into liquid nitrogen for storage. The samples were studied using a FEI Tecnai 12 G2 TEM, at 120 kV with a Gatan cryo-holder maintained at −180° C. Images were recorded on a slow scan cooled charge-coupled device CCD camera Gatan manufacturer and recorded with the Digital Micrograph software package, at low dose conditions, to minimize electron beam radiation damage.

LMW Biomarker Harvesting by PPAu Nanoparticles.

To test the ability of PPAu nanoparticles to harvest LMW biomarkers, SDFα, FITC-labeled (PK (FK)5PK, and PDGF B were incubated with fetal bovine serum (FBS) and PPAu nanoparticles as described below. Two methods were conducted to evaluate the PPAu nanoparticles harvesting performance: sodium dodecyl sulfate-polyacrylamide gel electrophoresis (SDS-PAGE) and fluorescence measurement.

SDS-PAGE.

In exemplary procedures, 10 μL of FBS (1:25 diluted in Tris-HCl 20 mM pH=7) and 10 μL SDFα (10 m/mL) at 20 mM of Tris-HCl pH=7) were incubated for 1 h. Then, 20 μL of PPAu (5.86×10″ particles/mL) nanoparticles were incubated with the above FBS+ SDFα solution for 1 h at RT. After incubation, samples were centrifuged at 10000×g for 10 min at 4° C. and washed three times, and the supernatant was saved. Particles and supernatant derived from particle incubation were loaded on 15% Tris-Gel. PPAu nanoparticles were stacked in the stacking gel while adsorbed proteins were eluted from the particles in resolving gel. Proteins' bands were detected using silver staining.

In exemplary procedures, 10 μL FBS (1:25 diluted in Tris-HCl 20 mM) and 10 μL of PDGF B (10 μg/mL at 20 mM of Tris-HCl pH=7) were incubated at RT for 1 h. Then, 20 μL of PPAu (5.86×10¹¹ particles/mL) nanoparticles were incubated with the above FBS+ PDGF B solution for 1 h at RT. After incubation, samples were centrifuged at 10000×g for 10 min at 4° C. and washed three times, and the supernatant was saved. Particles and supernatant derived from particle incubation were loaded on 15% Tris-Gel. PPAu nanoparticles were stacked in the stacking gel while adsorbed proteins were eluted from the particles in resolving gel. Proteins' bands were detected using silver staining.

To monitor the harvesting of SDFα in the presence of PDGF B by PPAu nanoparticles SDS-PAGE was applied. 10 μL FBS (1:25 diluted in Tris-HCl 20 mM) and 5 μL of each protein (0.625 μM, SDFα and PDGF B at 20 mM of Tris-HCl pH=7) was incubated at RT for 1 h. 20 μL of PPAu (5.86×10¹¹ NPs/mL) NPs were incubated with the above solution (FBS+SDFα+PDGF B) for 1 h at RT. After incubation, samples were centrifuged 10000×g at 4° C. and washed three times, and the supernatant was saved. Particles and supernatant derived from particle incubation were loaded on 15% Tris-Gel. PPAu nanoparticles were stacked in the stacking gel while adsorbed proteins were eluted from the particles in the resolving gel. Proteins' bands were detected using silver staining.

Fluorescent Measurement.

Triplicate of 20 μL FBS (1:25 diluted in 20 mM Tris-HCl) and 20 μL of FITC-labeled (PK (FK) 5PK) MW 2.219 kDa and pI 11.4 (10 μg/mL in 20 mM of Tris-HCl pH=7) were incubated together for 1 h at RT. Then, 40 μL of PPAu and naked Au NPs (5.86×10¹¹ NPs/mL) were incubated separately with the above FBS+ FITC-labeled (PK (FK) solution for 1 h at RT. After incubation, samples were centrifuged 10000×g at 4° C., and the supernatant was measured in 96-well micro-plates using an infinite M200 spectrophotometer (TECAN) equipped with a temperature controller set at RT, 485 nm to excitation and 519 nm emission. Triplicate of 50 μL of FBS (1:25 diluted in Tris-HCl 20 mM) of FITC-labeled (PK (FK) 5PK (10 μg/mL in 20 mM of Tris-HCl pH=7) were used as control for the fluorescent signal, and FBS was used as a background signal. A plot of the fraction of proteins bound to the NPs

$$\frac{{c}_{0} ( P ) - c ( P )}{{c}_{0} ( P )}$$

against the logarithmic initial protein concentration c₀ (P) allows fitting the data to the Hill equation:

$$\frac{{c}_{0} ( P ) - c ( P )}{{c}_{0} ( P )} = \frac{{N}_{max}}{1 + {( \frac{{K}_{D}^{′}}{c ( P )} )}^{n}} \frac{{c}_{0} ( NP )}{{c}_{0} ( P )}$$

where Nₘₐₓ is the maximum number of protein molecules which can be bound to each NP, K′_{D} is the supernatant protein concentration for which

$$\frac{{N}_{max}}{2}$$

of the protein binding sites of each NP are saturated. c₀ (NP) is the overall NP concentration, c(P) is the concentration of the unbound proteins, and n is the Hill coefficient.

Micro Bicinchoninic Acid (mBCA) Assay.

Triplicate of 40 μL of trypsin (MW 23.3 kDa, pI=10.4) at concentrations of (0.62×10⁻⁶M) and 40 μL PPAu NPs solution (5.86×10¹¹ NPs/mL) were incubated for 1 h at RT. After incubation, samples were centrifuged 10000×g at 4° C., and 50 μL of supernatant was measured by using mBCA assay according to the manual instructions. Triplicate of 50 μL of Tris-HCl pH=7 were used as control.

ELISA Measurements.

Triplicates of 100 μL FBS (1:25 diluted in Tris-HCl 20 mM) and SDFα at different concentrations (Exp. 1 (3.88 ng/mL) and Exp. 2 (1.88 ng/mL)) in 20 mM of Tris-HCl pH=7 were incubated together for 1 h at RT. Then, 50 μL of PPAu NPs (5.86×10¹¹ NPs/mL) were incubated with 50 μL of each FBS+ SDFα solutions for 1 h at RT. After incubation, samples were centrifuged 10000×g at 4° C., and the supernatant was saved. An amount of 50 μL of supernatant was measured by using ELISA kit (R&D systems) according to the manual instructions. Triplicates of 50 μL FBS +SDFα were used as control at each concentration. The concentration of the SDFα in the samples was estimated by a calibration curve which was constructed using known concentrations of SDFα stock solutions.

### Results

Monitoring SDFα Adsorption on PPAu NPs.

SDFα protein (MW=8 kDa, pI=10.3) was chosen as a LMW protein model to demonstrate its adsorption to PPAu NPs. SDFα is of particular interest because of its association to cancer: it promotes different mechanisms in cellular transformation and tumor growth such as angiogenesis, tumor spreading and metastasis.

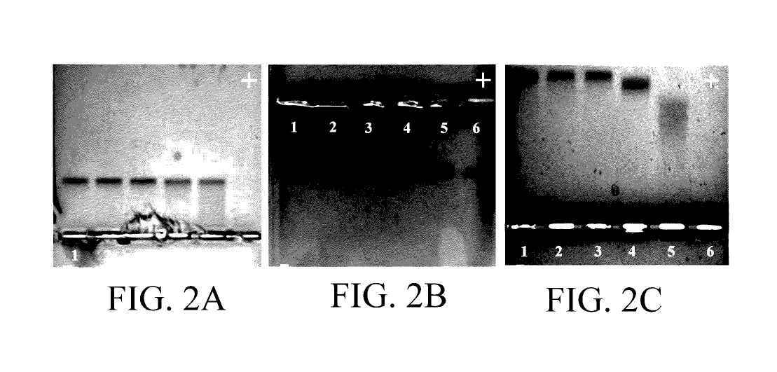

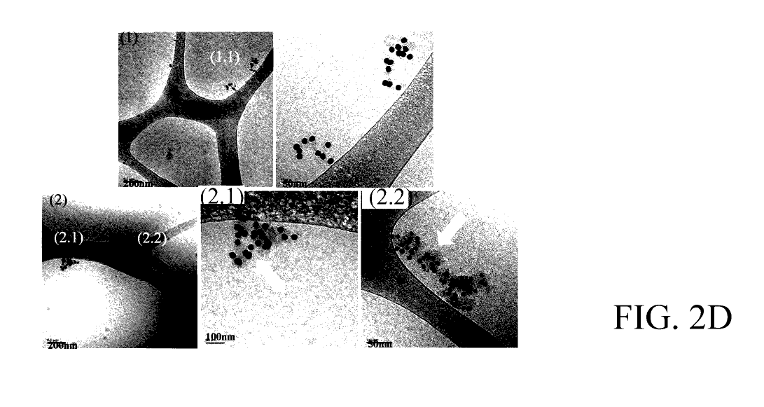

To monitor adsorption of SDFα on PPAu NPs, agarose gel electrophoresis (GE) (1%) was conducted (FIG. 2C). 20 μL of PPAu NPs (5.86×10¹¹ particles/mL) were incubated with various concentrations of SDFα solution (2.5 μg/mL-30 μg/mL) in 20 μL of 20 mM Tris-HCl (pH=7) for 1 h at room temperature (RT). Agarose gel electrophoresis results showed that PPAu NPs' band (FIG. 2C, lane 1) is clearly visible in the gel and shows larger mobility than that of PPAu NPs incubated with SDFα (2.5 μg/mL-30 μg/mL) in lanes 2-6 (FIG. 2A). The mobility reductions in lanes 2-6 are attributed to the particles' size increase and to the potential decrease, or to the Debye length increase when the size and charge are unchangeable. SDFα adsorption on the PPAu NP surface weakens the electrostatic repulsion between the NPs while allowing the van der Waals attraction to become dominant and generate larger aggregates. In addition, the decrease in the ζ-potential reduces the driving force acting on the NPs. As a result, a broader distribution of NP sizes is obtained as reflected by the band smear in lane 5.

In FIG. 2C lane 6, PPAu-SDFα complexes remained stuck in the gel well, probably because of the formation of large aggregates and the reduction of the ζ-potential. However, incubation of naked Au and PEI-Au NPs with various concentrations of SDFα did not lead to mobility differences between bands as shown in FIG. 2B, and slight mobility differences between bands in FIG. 2A. SDFα either escaped from the gel due to its small size or adsorbed to the NPs' surfaces (FIG. 2A, lane 6). This suggested that SDFα did not adsorb to PEI-Au or to the surface of naked Au NPs as in PPAu NPs. It was therefore concluded that PAA was the cause for SDFα adsorption. The aggregate hypothesis was supported by Cryo-TEM images of samples used in the GE showing that the PPAu NPs were enclosed within a protein SDFα matrix designated by the arrow in FIG. 2D. Notably, larger aggregates were formed by increasing the SDFα concentration. Other studies on the effects of protein adsorption on Au NPs showed similar formations of protein-NP complexes.

LMW Biomarker Harvesting by PPAu Nanoparticles.

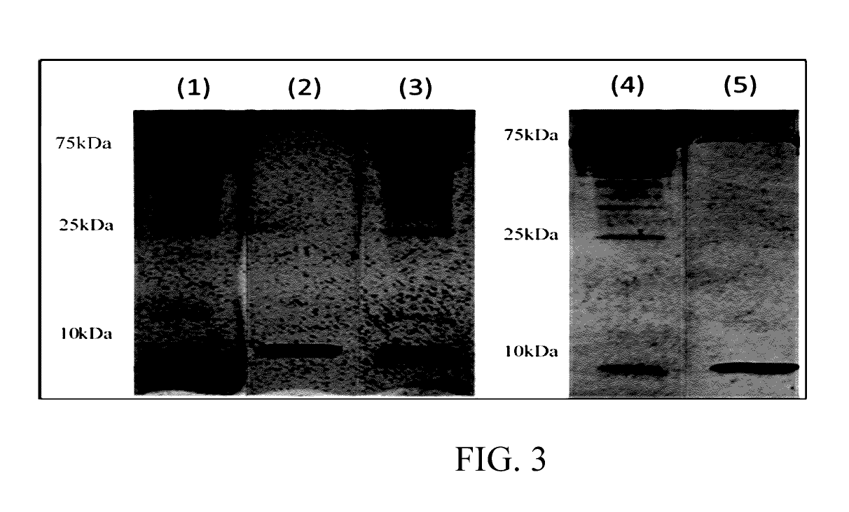

Two independent methods were applied to assess the harvesting potential of PPAu NPs: sodium dodecyl sulfate-polyacrylamide gel electrophoresis (SDS-PAGE) and fluorescence measurements. In the first method, SDFα (10 μg/mL) was incubated with fetal bovine serum (FBS) (50%) for 1 h at room temperature (RT). FBS was chosen because it contains more than 3700 positively and negatively charged proteins and therefore simulates an in vivo situation. After SDFα incubation with FBS, PPAu NPs (5.86×10¹¹ NPs/mL) were added to the solution, and incubated for 1 h at RT, then washed and centrifuged three times. The NPs and supernatant were both loaded on the gel, demonstrating the NPs' capacity to harvest and concentrate most of the SDFα and separate most of it from the rest of the solution in one step (FIG. 3: lanes (4) and (5)), leaving out all the BMW proteins. From lane (1), we can see that FBS has several HMW proteins that exist in high concentrations (e.g. albumin 66 kDa, IgG 25 kDa).

In the second fluorescence-based method PPAu and naked Au NPs dispersions were incubated separately with FITC-labeled peptide (PK (FK) 5P), MW 2219.70 Da and pI=11.50 which was previously incubated with FBS, then washed and centrifuged three times as in the former method.

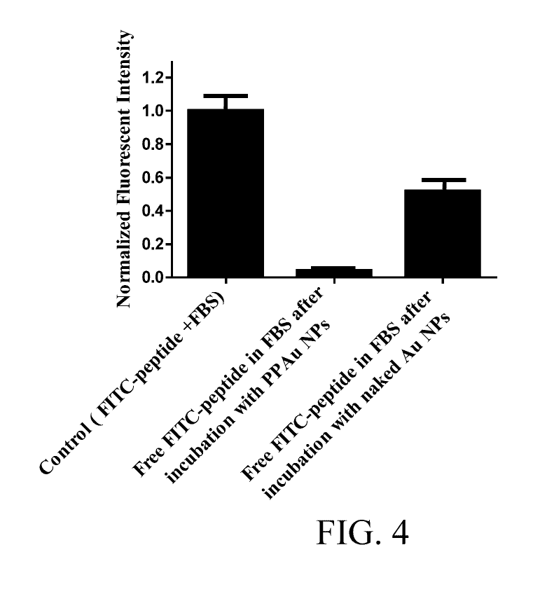

The fluorescent intensity of the supernatant was measured using fluorometer. The FITC-peptide in FBS served as control for the fluorescent signal, and the FBS served as control for the background signal. It can be seen (FIG. 4) that the remaining peptide in the supernatant is significantly less (˜4%) than its initial amount in the control after incubation with PPAu NPs. On the other hand, in the case of naked Au NPs, approximately half of FITC-peptide is free in the sample. These results indicate that FITC-labeled peptide has larger affinity to PPAu than to naked Au NPs suggesting the significant reduction in the fluorescent intensity is due to binding on NPs surface and not as a result of FITC molecule quenching by gold. The reduction in fluorescence intensity of naked Au NPs is not surprising given the charge of naked Au NPs (FIG. 4).

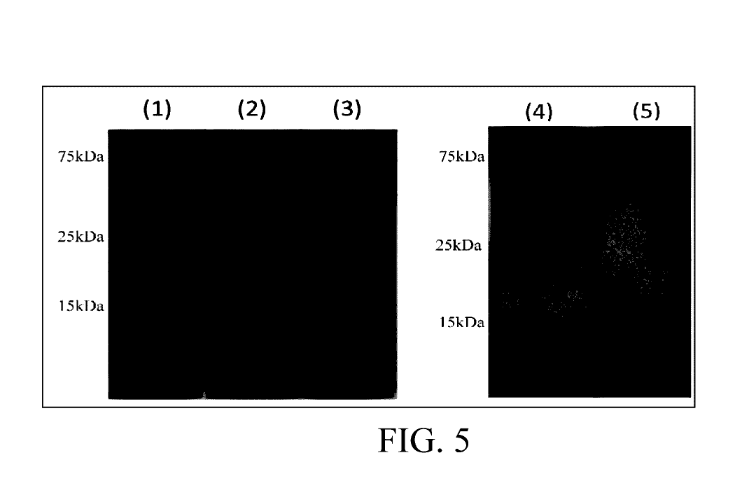

To assess the selectivity of PPA NPs to LMW biomarkers (<10 kDa), Platelet Derived Growth Factor B (PDGF B) (14.4 kDa, pI=9.4) was chosen as a competing, positively-charged model biomarker. PDGF B was incubated with FBS for 1 h at RT and then PPAu NPs were added to the solution. The solution was washed and centrifuged three times after which the PPAu NPs and the supernatant were loaded on gel lanes. As seen in FIG. 5, a minor amount (˜30%) of the entire PDGF B is harvested by PPAu NPs (lane 5), indicating that the affinity of PPAu to proteins with molecular weight (MW) higher than 10 kDa is low compared to that of SDFα and FITC-labeled peptide which were totally harvested from the serum.

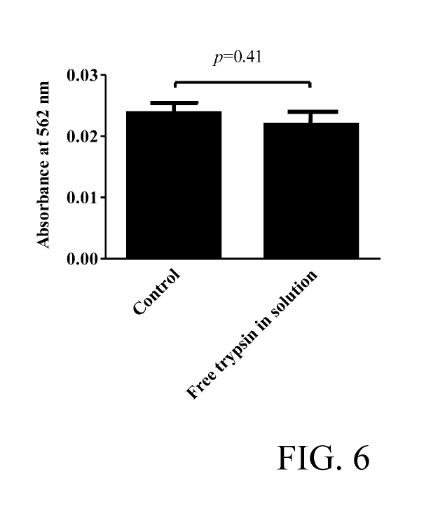

An experiment was conducted to demonstrate the selectivity of PPAu NPs to LMW cationic peptides. Trypsin solution at a concentration of 0.62×10⁻⁶M was incubated with PPAu NPs (5.86×10¹¹ NPs/mL) for 1 h at RT. Then, the dispersion was centrifuged and the supernatant was saved. The concentrations of free trypsin in the solution and in the control samples were quantified using Micro Bicinchoninic Acid (mBCA) assay. In FIG. 6 it can be seen that the concentration of free trypsin in the solution after incubation with PPAu NPs has not changed compared to the control sample of trypsin (p=0.41). This suggests that the selective harvesting property of the PPAu NPs is a resultant of two factors: their electric charge and their attraction to LMW proteins. It is hypothesized, without being bound by any particular theory, that fine-tuning the combinations of these factors may increase the resolution of the captures and provide reliable diagnostic tools.

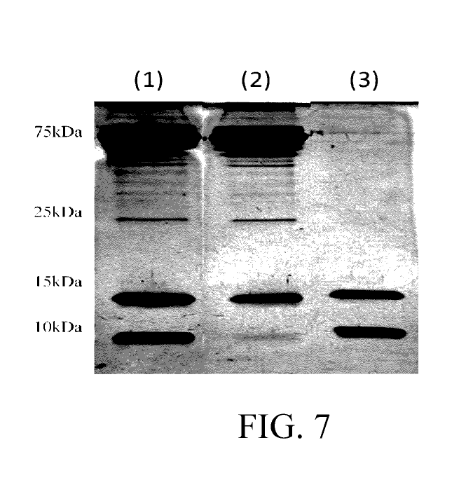

Another experiment was conducted to assess the harvesting potential of PPAu NPs in the presence of two different positively charged proteins and FBS. SDFα, PDGF B and FBS were incubated together, and then PPAu NPs were added to the solution. The SDS-PAGE procedure was carried out as described above. In FIG. 7 at lane 3 it can be seen that PPAu NPs harvest most of the SDFα in the solution and only a minute fraction of the PDGF B. The remaining FBS proteins and PDGF B are left in the supernatant (lane 2) indicating that PPAu NPs harvest SDFα and LMW proteins from the serum selectively and effectively.

More experiments were performed to assess the harvesting sensitivity of PPAu NPs at an extremely low concentration of SDFα in a mock diseased state. SDFα was incubated with FBS: [3.88 ng/mL] in the first experiment (Exp. 1) and [1.88 ng/mL] in the second experiment (Exp. 2). Then, PPAu NPs (5.86×10¹¹ NPs/mL) were added to the solution and were incubated for 1 h at RT. The entire dispersion was centrifuged and the supernatant was saved. The enzyme-linked immunosorbent assay (ELISA) procedure was used to evaluate the SDFα concentration in the supernatant and in the control samples.

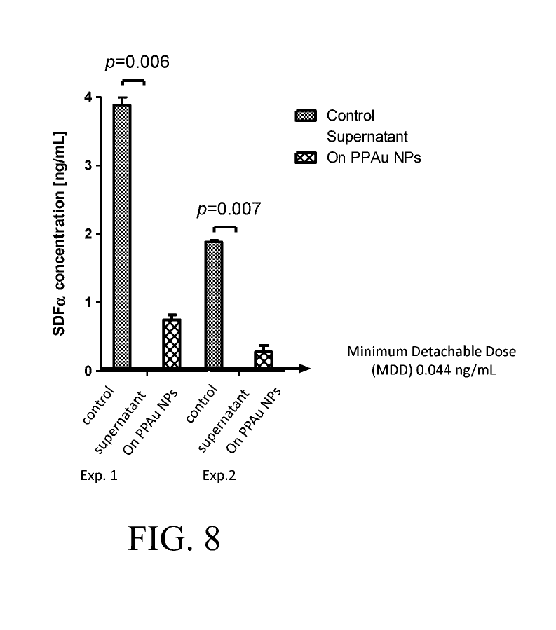

In FIG. 8, Exp. 1, it can be seen that the concentration of SDFα in the supernatant was 3.13±0.076 ng/mL compared to the control sample (3.88±0.198 ng/mL, p=0.006). This suggests that the remaining SDFα protein (0.75±0.123 ng/mL) had adsorbed to the PPAu NPs' surfaces-which is above the detection threshold of the ELISA kit at hand (0.044 ng/mL). At the lower concentration of SDFα (1.88 ng/mL) in Exp. 2 the concentration in the supernatant was 1.60±0.169 ng/mL compared to that in the control sample (1.88±0.04 ng/mL, p=0.007), suggesting that the remaining SDFα protein had adsorbed to the PPAu NPs' surfaces.

In clinically-relevant cases, after separation of the LMW proteins/peptides, the harvested molecules eluted from the PPAu NPs or the supernatant can be analyzed using other sensitive methods such as MALDI-TOF (matrix assisted laser desorption ionization time-of-flight), which can identify and classify all LMW proteins/peptides. The disclosed approach is superior to conventional immunoassay platforms, such as antibody arrays and antibody-conjugated NPs which cannot effectively measure panels of analyte fragments.

Without being bound by any particular theory, it is assumed that this selectivity may be attributed to the nanoparticle surface curvature, and to the functionalizing group on the PPAu nanoparticle surface.

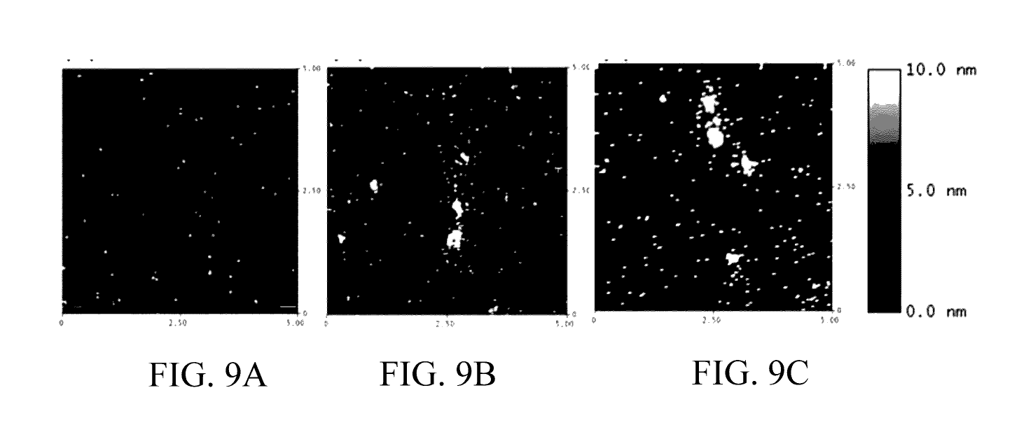

Atomic Force Microscopic (AFM) examination results presented in FIGS. 9A-C, demonstrated a significant difference between naked PPAu nanoparticles (FIG. 9A) and complexes of SDFα (0.02 mg/mL and 0.03 mg/mL) and PPAu nanoparticles (FIGS. 9B and 9C, respectively). These differences arise from the fact that single positively charged SDFα molecules can be simultaneously by several different nanoparticles, which lead to PPAu nanoparticle aggregates proportional in size to the protein concentration.

Zeta Potential.

After incubation of PPAu nanoparticles with SDFα, their ζ potential increased from −55 mV to −12 mV.

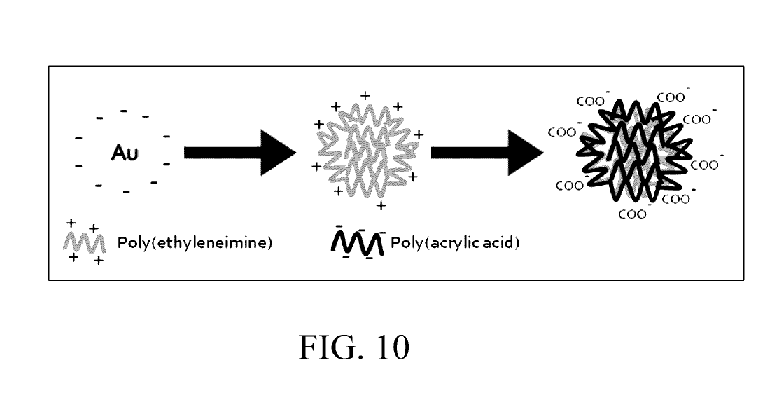

FIG. 10 presents, to summarize some embodiments of the invention, schematic description of the layer-by-layer (LbL) strategy to functionalize Au nanoparticles according to some embodiments of the invention.

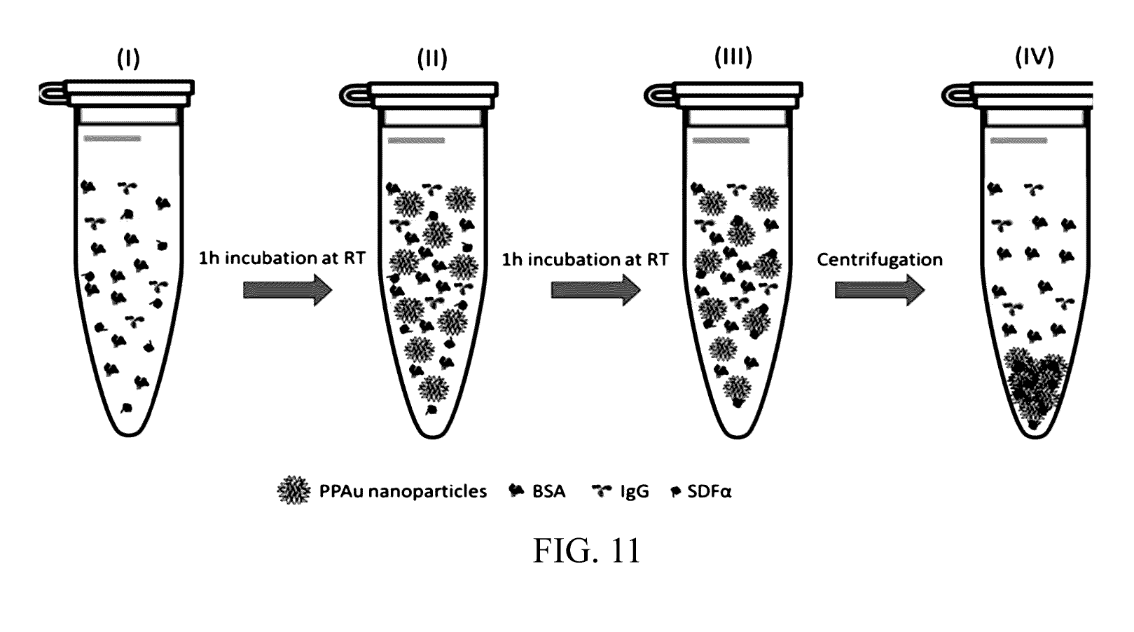

FIG. 11 presents a schematic illustration of harvesting SDFα from FBS by PPAu NPs according to some embodiments of the invention. (I) LMW biomarker incubated with FBS; (II) PPAu NPs added to FBS and LMW biomarker; (III) LMW biomarker adsorbed on PPAu NPs; (IV) PPAu NPs separated by centrifugation from the dispersion for analysis.

### Example 4

### Monitoring the Adsorption of Various Proteins on Naked (Au) and on Coated (PPAu) NPs: A Comparative Study

UV-Vis.

To monitor the adsorption of various proteins with different molecular weights (Table 1 below) to the surface of naked (Au) and coated (PPAu) NPs, UV-visible spectra were used, as described hereinabove.

In exemplary procedures, 50 μL of naked (Au) or coated (PPAu) NP dispersions were incubated for 1 h at RT with 50 μL of various solutions of proteins/peptides in Tris-HCl 20 mM pH=7: ((i) PK(FK)5PK, (ii) LL-37 peptide, (iii) cationic trypsin, and (iv) BSA. The samples underwent spectral analysis in the 450-700 nm range. Naked Au and PPAu NPs in double distilled water (DDW) and Tris-HCl (20 mM pH=7) were used as controls.

CryoTEM.

Naked Au and PPAu (5.86×10¹¹ NPs/mL) were incubated for 1 h at RT with various proteins/peptides in Tris-HCl 20 mM pH=7: ((i) PK(FK)5PK (436.8 μM), (ii) LL-37 (222.5 μM), (iii) cationic trypsin (6 μM), and (iv) BSA (13.8 μM)). Samples were prepared as described hereinabove.

Interaction of NPs with Proteins More Complex Environment:

To understand the influence of NP surface heterogeneity in a more complex environment, the interaction between naked Au and PPAu NPs with four different proteins (Table 1) were studied by UV-vis, cryoTEM, and Hill model.

TABLE 1

| | | Molecular Weight | |
|---|---|---|---|
| # | Protein Model | (kDa) | pI |
| (i) | PK(FK)5PK | 1.9 | 11 |
| (ii) | LL-37 | 4.49 | 11.1 |
| (iii) | Cationic Trypsin | 23.3 | 10.3 |
| (iv) | BSA | 67 | 5.9 |

BSA.

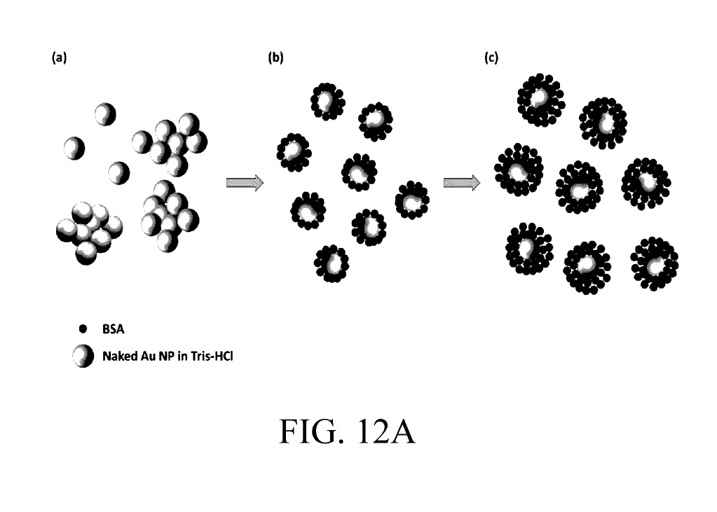

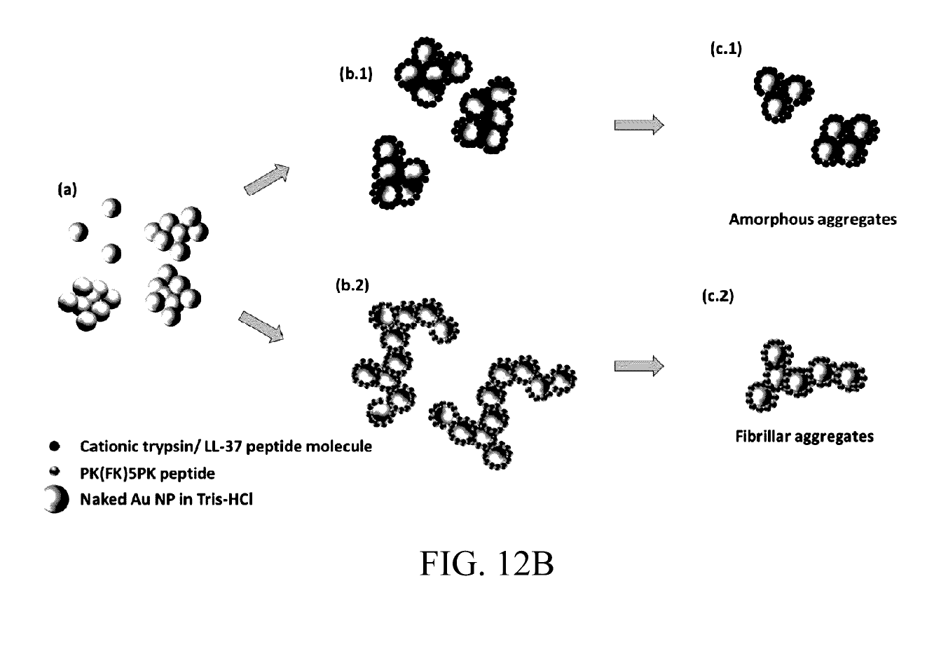

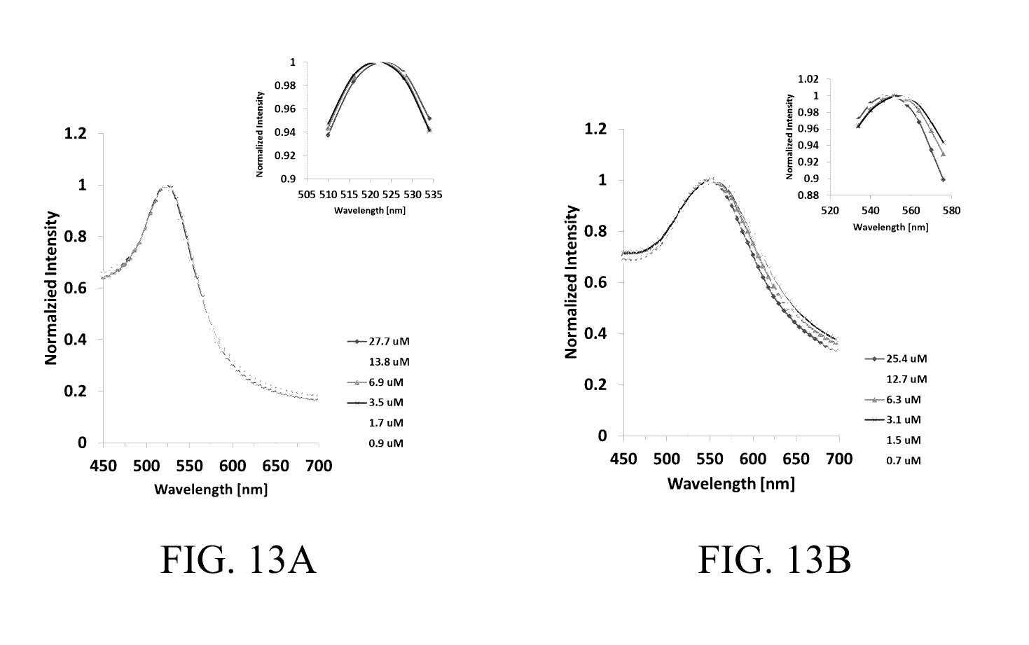

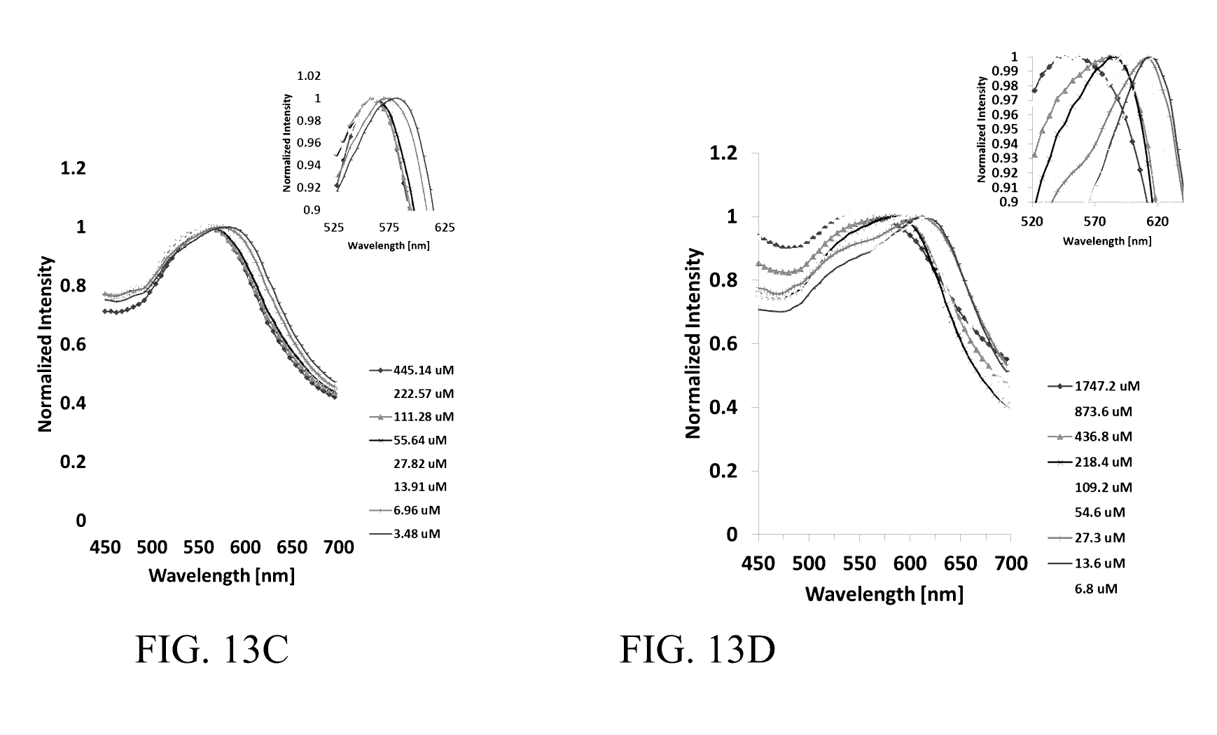

When a sufficient amount of BSA was added to a dispersion of Au NPs in Tris-HCl (>30 nmol/L) their aggregation was reversed (as demonstrated in the scheme of FIG. 12A) and the resulting UV-vis spectrum wavelength (FIG. 13A) appeared similar to that of naked Au NPs in water, or in Tris-HCl less the 2^{nd} broad band centered at ˜630 nm in FIG. 1F.

FIG. 12B presents schematic illustration of the formation of protein-Au NPs complexes: (a) Naked Au NPs' aggregates in Tris-HCl (20 mM); (b) protein-Au NPs aggregates: (1) cationic trypsin or LL-37 peptide at low concentration, (2) PK(FK)5PK peptide at low concentration; (c) typical morphologies of protein-Au NPs aggregates obtained at higher protein concentrations: (1) cationic trypsin or LL-37 peptide, (2) PK(FK)5PK peptide.

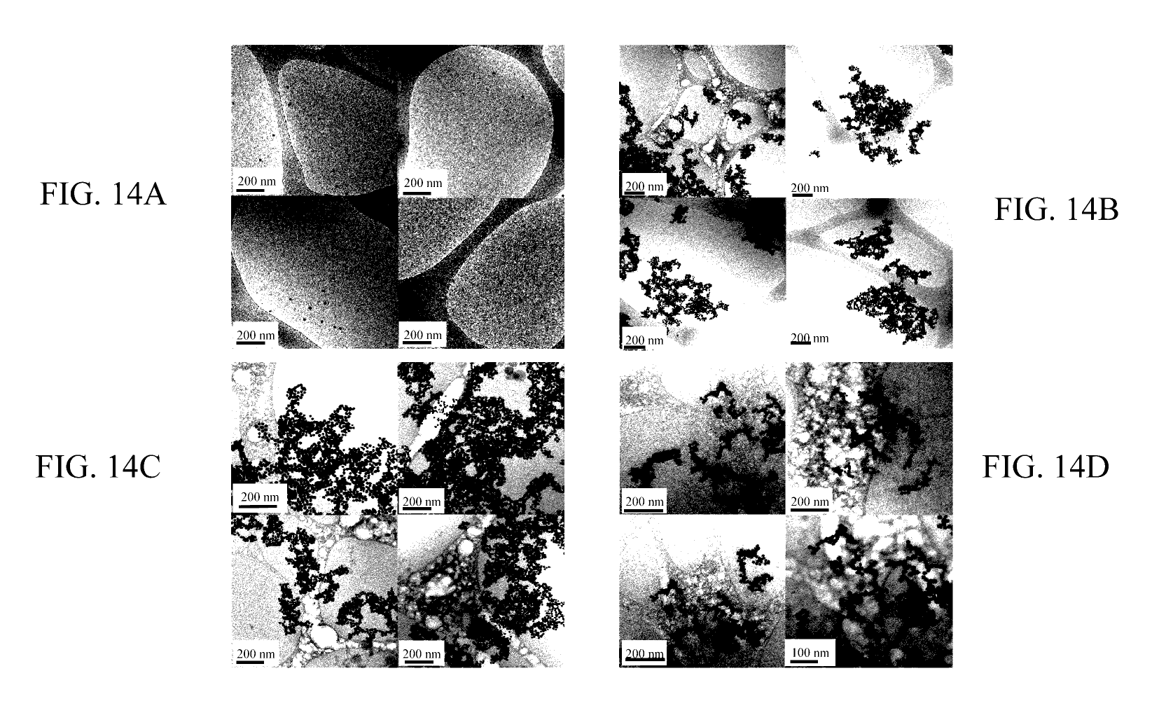

These results are corroborated by the cryoTEM images in FIGS. 14A-D. However, the bands incrementally shifted to red as the BSA concentration in the dispersion was increased, indicating the thickening of the BSA layer on the NPs' surface while maintaining the NPs' stability (FIG. 14A). It might seem odd at first that citrate-capped Au NPs adsorb BSA (pI=4.8) molecules because at pH=7 both: the citrate and the BSA are negatively charged however BSA has 60 surface lysine residues that can be protonated and interact electrostatically with the negatively charged NPs. Furthermore, other forces such as hydrophobic interactions may have a role in the conjugation of the BSA with the Au NPs' surface.

As noticed in FIG. 13B, addition of cationic trypsin to naked Au NPs disassembled aggregates and led to UV-vis blue shift. This phenomenon can be attributed to the layer of cationic trypsin formed on each naked Au NP, inducing electrical repulsion and leading to disassembly of aggregates and improved NPs' stability. It is noticed that the spectral peak of NPs with trypsin, beginning at a wavelength of 550 nm for trypsin concentration of 0.7 μM decreases monotonically with trypsin concentration, i.e. blue shift (FIG. 13B). It is speculated that due to their smaller size more cationic trypsin molecules than BSA molecules are required to fully cover the NP surface area. They might act as spacers and cause steric separation or accumulate sufficient electric repulsion between the NPs causing aggregate disassembly, as reflected by UV-Vis blue shift.

In addition, as seen in FIG. 14B the aggregates resulting from Au NPs and cationic trypsin (6 μM) are smaller than those of Au NPs in Tris-HCl. This observation may also explain the absence of a second UV-vis band in the case of naked Au+cationic trypsin (FIG. 13B), compared to the one that appears (centered at 630 nm) in naked Au+Tris-HCl (FIG. 1F).

A similar behavior as with cationic trypsin (FIG. 13B) is seen FIG. 13C, and FIG. 13D upon the addition of the low molecular weight peptides LL-37 (4.5 kDa, pI-10.4) and PK(FK)5PK (1.9 kDa, pI˜11.2), respectively to a dispersion of naked Au NPs. These peptides' sizes are proportional to their molecular weight, respectively covering a smaller portion of the NP surface area, therefore more of these molecules are needed to form a shielding layer that promotes aggregates' disassembly as in the case of cationic trypsin.

FIG. 14C presents cryo-TEM images of amorphous aggregates formed after 1 h incubation of Au NPs and LL-37 peptides. On the other hand, with PK(FK)5PK peptides and Au NPs the aggregates remain fibrillary even at a high peptide concentration (FIG. 14D). These patterns may be attributed to the differences between the peptide types.

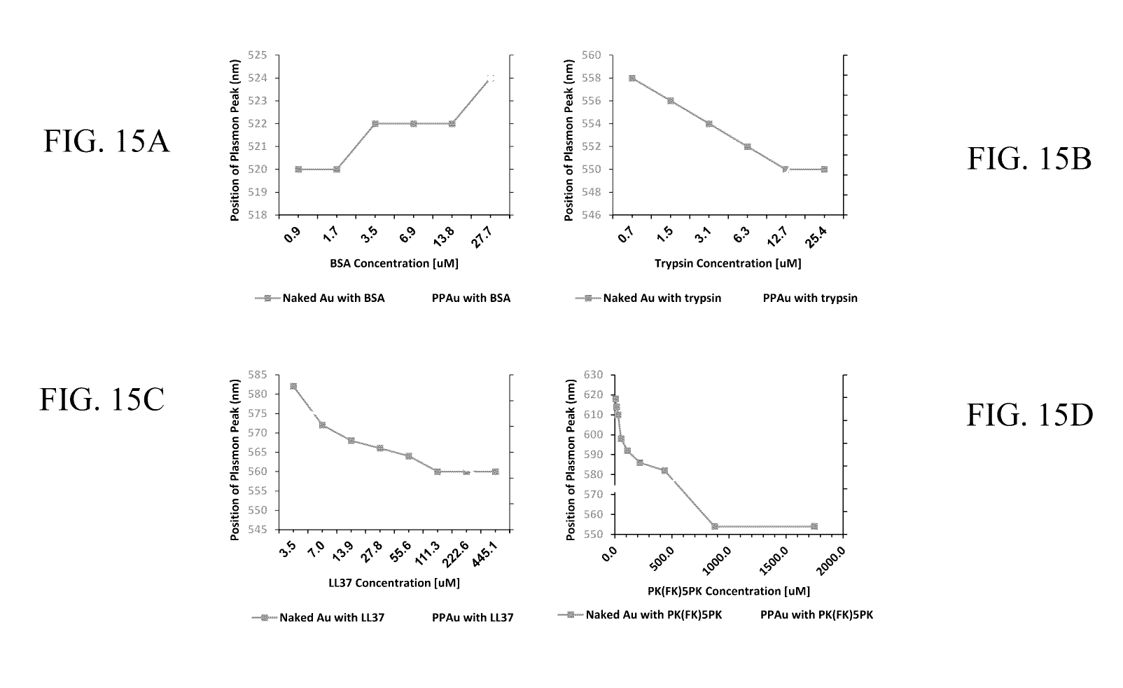

FIGS. 15A-D present the position of plasmon peak versus protein concentration for naked Au and PPAu NPs with BSA (FIG. 15A); cationic trypsin (FIG. 15B); LL37 peptide (FIG. 15C); and PK(FK)5PK peptide (FIG. 15D). As shown in FIGS. 15A-D red shift was observed when naked Au NPs incubated with BSA, and blue shift when naked Au NPs incubated with cationic trypsin, LL37 peptide, and PK(FK)5PK peptide. On the other hand, no shifts were observed resulting from incubation of PPAu NPs with BSA and cationic trypsin, while blue and red shift observed when PPAu NPs incubated with LL37 peptide and PK(FK)5PK peptide, respectively.

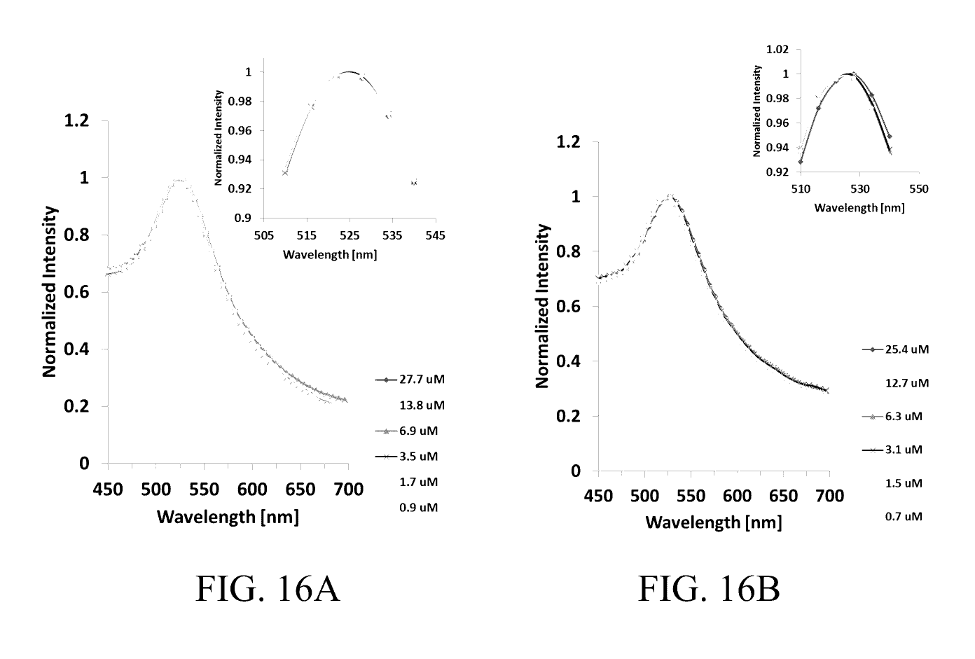

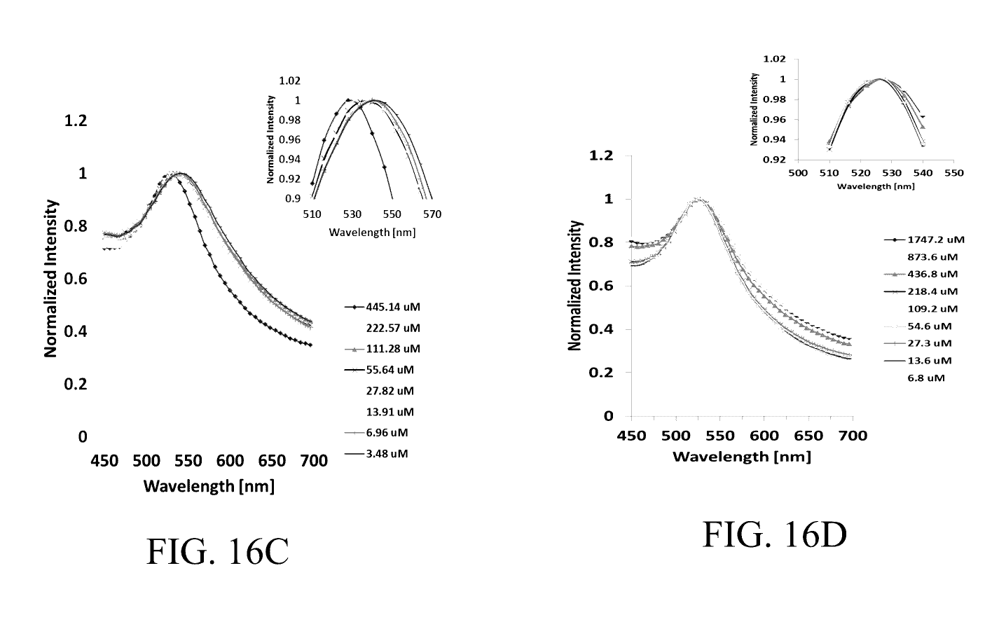

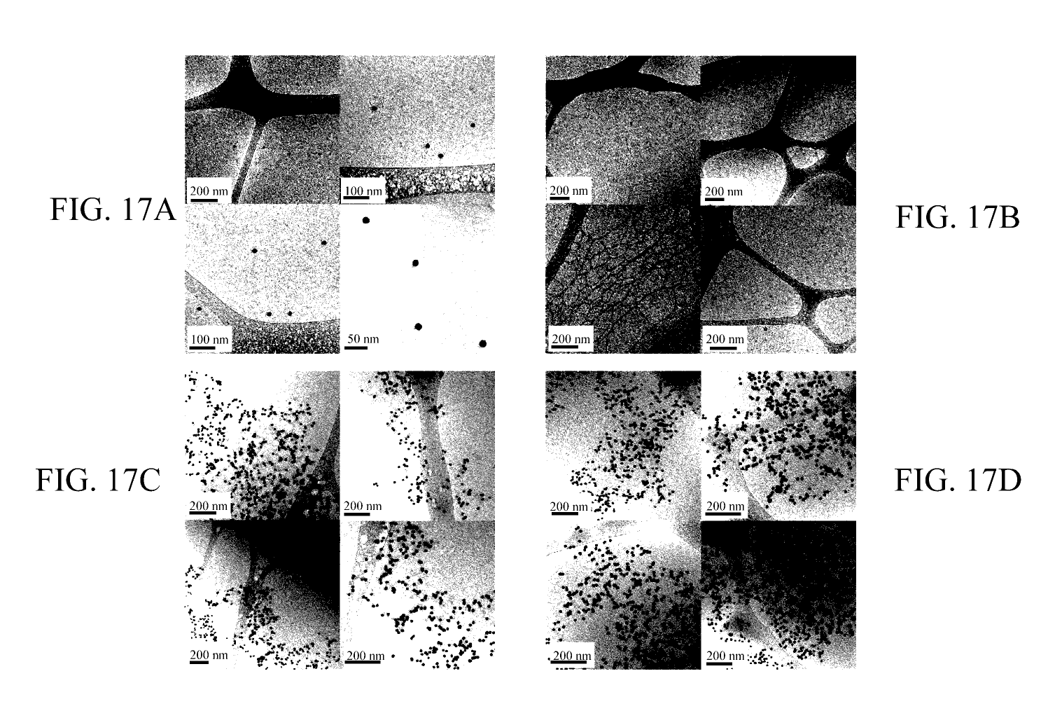

When PPAu NPs were incubated with BSA or cationic trypsin, no shifts were observed in the wavelength spectrum in response to any concentration changes (FIGS. 16A, B). These results suggest that BSA and cationic trypsin did not adsorb onto the PPAu surfaces. These results were also corroborated by cryoTEM images (FIGS. 17A, B) showing no NP agglomeration (unlike the case of naked Au NPs).

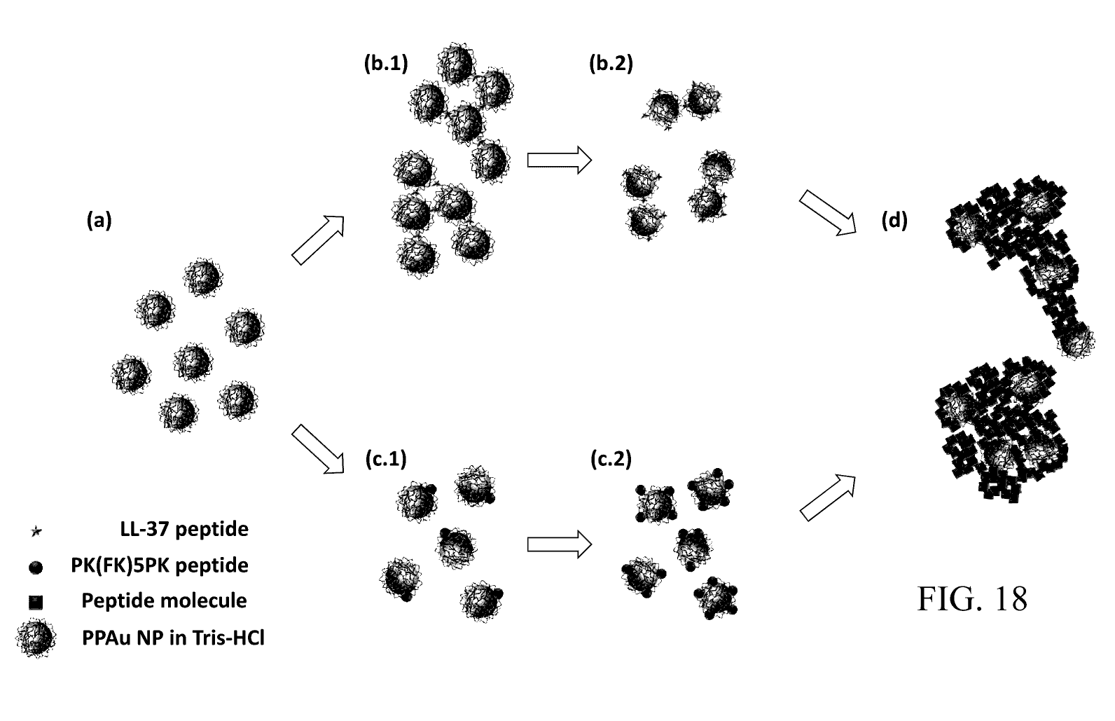

FIG. 16C demonstrates that incubation of PPAu NPs with low concentration (3.48 μM) of positively charged LL-37 peptide led to a red shift in the spectral curves compared to PPAu in Tris-HCl (FIG. 1F), while further increasing the concentration of LL-37 peptide led to a blue shift. This behavior could indicate that proteins at low concentration may link together few PPAu NPs (as illustrated in the scheme of FIG. 18), while further increasing the peptide concentration enhances steric and electric separation between PPAu NPs due to PPAu surfaces' coverage and the creation of smaller aggregates (as illustrated in scheme 3b.2 in FIG. 18). Upon further increase of the peptide concentration the smaller aggregates serve as building blocks of yet larger, globular or fibril aggregates (scheme 3d in FIG. 18) and in the presence of the PK(FK)5PK peptide, respectively.

It can be seen that when LL-37 (222.5 μM) was incubated with PPAu NPs, the PPAu NPs were enclosed within a LL-37 matrix in small aggregates, as shown FIG. 17C. These observations were also shown in several studies which discussed similar formations of protein-NP complexes.

It is to note that when PK(FK)5PK peptide is incubated with PPAu NPs (FIG. 16D), increasing the concentration of the peptide, slightly led to a red shift and widening of the spectral curves indicating that the peptide adsorbed onto the PPAu NPs' surface. These results are also corroborated by the cryoTEM images presented in FIG. 17D showing the behavior of PPAu NPs in the absence of the peptide.

It is speculated, without being bound by any particular theory, that the difference between the interaction of naked Au and PPAu NPs with BSA can be attributed to the difference in their NP surface morphology, because both NP types are of nearly the same size and negatively charged. While both PPAu and naked Au NPs have similar carboxyl groups on their surface, their surface structure and morphology differ due to the LbL coating method of the PPAu NPs. These differences might hinder the adsorption of proteins onto the PPAu NPs by preventing the proteins from direct (or tight) contact with the Au surface.

Contrary to BSA, cationic trypsin is positively charged and hence was expected to adsorb onto both types of NPs owing to electrostatic attraction. Surprisingly, cationic trypsin adsorbed only onto naked Au NPs. Again, this behavior could have been caused by the high adsorbing affinity of proteins to flat and smooth surfaces. The surface and morphology changes resulting from the coating method could affect the curvature and roughness of the PPAu NP's surface and hinder the adsorption of the larger cationic molecules of trypsin onto the PPAu NP's in spite of its charge.

Hill Model.

Using the equation hereinabove, the fraction of protein adsorbed onto NPs' surfaces,

$$( \frac{{c}_{0} ( P ) - c ( P )}{{c}_{0} ( P )} )$$

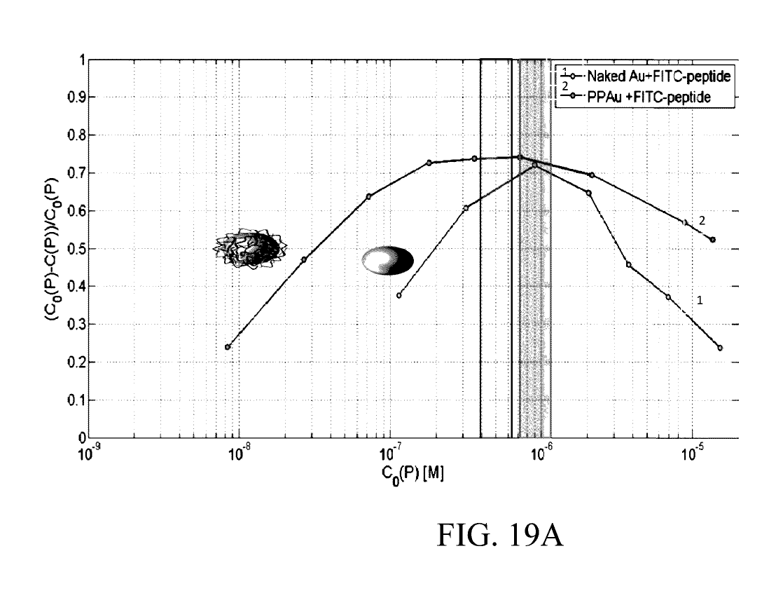

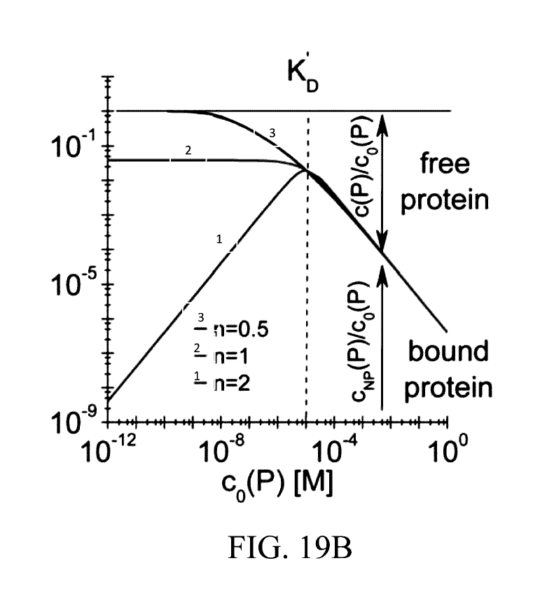

was calculated and drawn as a function of c₀ (P), where C₀ (P) is the initial protein concentration, and c (P) is the concentration of the unbound proteins. It can be seen FIGS. 19A-B that K′_{D}, (the supernatant protein concentration for which half of the protein binding sites on each NP are saturated) in both: PPAu and naked Au NPs are of the same order of magnitude (green and blue rectangles), approximately 10⁻⁷M. In addition, the results shown in FIG. 19A indicate that the Hill coefficient n is greater than 1 as described in Pino et at (Mater. Horiz. 2014, 1, 301; FIG. 19B). For C₀ (P)<K′_{D} cooperative adsorption (n>1) leads to fewer bindings compared to uncooperative adsorption; with cooperative adsorption it is easier for protein molecules to bind simultaneously to NPs rather than separately. At low protein concentration the probability of simultaneous protein bindings to each NP is low resulting in fewer bindings. The simultaneous binding probability increases with increasing initial protein concentration as seen in FIGS. 19A-B. High initial protein concentration (c₀ (P)>>K′_{D}) imposes saturation of the available binding sites and the effect of n decays because n is relevant only for large numbers of empty sites on the NP surface. It is noted that the domain of dependence corresponding to the naked Au NPs is narrower than that of PPAu NPs (leading to a steeper curve). This characteristic is attributed to the NP aggregation and the associated reduction of available surface area of the naked Au NPs for peptide adsorption.

In order to improve the harvesting efficacy of the PPAu NPs for lower protein concentration, higher NP concentration C₀ (NP) is needed. Elevating the NP concentration in the solution increases the probability for each protein molecule to adsorb onto the surface of NP. It appears that the PPAu NPs could also be used to effectively remove the above proteins from a solution, with possible purification applications.

It can be concluded that among the main properties determining the adsorption profile of protein corona on the NP surface are its surface chemical composition and structure. In this study, we studied the adsorption of several proteins with different molecular weight to negatively-charged naked Au NPs of size comparable to that of PPAu NPs. The adsorption and the stability of the NPs was investigated and analyzed by combining cryoTEM, UV-vis, and the Hill equation. The results showed that the protein adsorption behavior depended on the chemical composition and the morphology of the NPs' surface and that PPAu NPs selectively adsorbed LMW proteins and peptide. This study suggests that NP surface structure and composition might be tailor-fitted for specific applications such as: bio-separation and LMW protein harvesting.

Although the invention has been described in conjunction with specific embodiments thereof, it is evident that many alternatives, modifications and variations will be apparent to those skilled in the art. Accordingly, it is intended to embrace all such alternatives, modifications and variations that fall within the spirit and broad scope of the appended claims.

All publications, patents and patent applications mentioned in this specification are herein incorporated in their entirety by reference into the specification, to the same extent as if each individual publication, patent or patent application was specifically and individually indicated to be incorporated herein by reference. In addition, citation or identification of any reference in this application shall not be construed as an admission that such reference is available as prior art to the present invention. To the extent that section headings are used, they should not be construed as necessarily limiting.

What is claimed is:

1. Nanostructure being negatively charged, said nanostructure comprises at least one gold nanoparticle, wherein said at least one gold nanoparticle:
(a) bears on at least portion thereof a positively charged polymer selected from polyethylenamine (PEI), cationic polyallylamine, poly-L-lysine, poly(allylamine hydrochloride), and poly(diallyldimethylammonium chloride) or any derivative thereof, wherein said positively charged polymer bears on at least portion thereof a negatively charged polymer selected from poly(acrylic acid) (PAA), polystyrenesulfonate, polyvinyl sulfate, polyvinylsulfonic acid or any derivative thereof; and
(b) has a diameter of between 5 and 20 nm,
and wherein said nanostructure has a hydrodynamic diameter of less than 100 nm.

2. The nanostructure of claim 1, having a hydrodynamic diameter of less than 25 nm.

3. The nanostructure of claim 1, wherein said positively charged polymer has an average molecular weight (MW) that ranges from 6 kDa to 15 kDa.

4. The nanostructure of claim 1, characterized by a zeta-potential of at least |−30| mV in aqueous dispersion.

5. The nanostructure of claim 1, characterized by a high affinity to a positively charged low molecular weight (LMW) molecules.

6. The nanostructure of claim 5, wherein said LMW molecules are selected from peptides, proteins, platelet-derived microparticles, and apoptotic bodies.

7. The nanostructure of claim 5, wherein said LMW molecules have a MW value of less than 15 kDa.

8. The nanostructure of claim 5, wherein said LMW molecules are selected from Stromal Derived Factor-alpha (SDF-alpha) and Platelet Derived Growth Factor B.

9. A composition comprising a plurality of the nanostructures of claim 1.

10. The composition of claim 9, wherein at least 90% of said plurality of the nanostructures are characterized by an averaged diameter that varies within less than ±20%.

11. The composition of claim 9, for use in detection of positively-charged low molecular weight (LMW) molecules.

12. The composition of claim 9, for use in diagnosis and/or detection of medical disorder or disease, said medical disorder or disease being characterized by occurrence of excessive positively charged low molecular weight (LMW) molecules.

13. The composition of claim 12, wherein said disease is cancer and/or inflammatory-disease.

14. The composition of claim 12, wherein said detection is in a sample of biological cells, in vitro, ex vivo, in vivo, or for clinical imaging.

15. The composition of claim 12, further comprising an agent selected from radiolabel, an X-ray contrast agent, a magnetic resonance imaging (MRI) contrast agent, and a fluorescent label.

16. The composition of claim 12, being detected by a method selected from fluorescent microscope, a flow-cytometric equipment, MM contrast, X-ray or computerized tomography (CT) contrast.

17. A method for the detection of positively charged LMW molecules in a sample, the method comprising:
(i) contacting the sample with a composition comprising a plurality of nanostructures under conditions enabling binding of nanostructures to positively-charged LMW molecules; and
(ii) detecting bound nanostructures to said LMW molecules, thereby indicating the presence of positively-charged LMW molecules,
wherein said nanostructures:
(a) are negatively charged, (b) comprise at least one gold nanoparticle, said at least one gold nanoparticle:
(i) bears on at least portion thereof a positively charged polymer selected from polyethylenamine (PEI), cationic polyallylamine, poly-L-lysine, poly(allylamine hydrochloride), and poly(diallyldimethylammonium chloride) or any derivative thereof, wherein said positively charged polymer bears on at least portion thereof a negatively charged polymer selected from poly(acrylic acid) (PAA), polystyrenesulfonate, polyvinyl sulfate, polyvinylsulfonic acid or any derivative thereof; and
(ii) has an average diameter of between 5 and 20 nm,
and wherein said nanostructures have an average hydrodynamic diameter of less than 100 nm.

18. The method of claim 17, wherein said sample is selected from blood serum and blood plasma.

19. The method according to claim 17, being performed by a kit, the kit comprising a medium having affixed thereto said nanostructure and a detecting assay for detecting LMW molecules.

20. A method for the detection of a physiological disorder or disease characterized by the presence positively-charged LMW molecules, the method comprising:
(i) administering a composition comprising a plurality of nanostructures to a patient in need thereof; and
(ii) imaging the patient using an appropriate imaging technique.
wherein said nanostructures:
(a) are negatively charged, (b) comprise at least one gold nanoparticle, said at least one gold nanoparticle:
(i) bears on at least portion thereof a positively charged polymer selected from polyethylenamine (PEI), cationic polyallylamine, poly-L-lysine, poly(allylamine hydrochloride), and poly(diallyldimethylammonium chloride) or any derivative thereof, wherein said positively charged polymer bears on at least portion thereof a negatively charged polymer selected from poly(acrylic acid) (PAA), polystyrenesulfonate, polyvinyl sulfate, polyvinylsulfonic acid or any derivative thereof; and
(ii) has an average diameter of between 5 and 20 nm,
and wherein said nanostructures have an average hydrodynamic diameter of less than 100 nm.
